(19) United States
(12) Patent Application Publication
(10) Pub. No.: US 2024/0288307 A1
(43) Pub. Date: Aug. 29, 2024

(54) SYSTEMS AND METHODS TO ACQUIRE THREE DIMENSIONAL IMAGES USING SPECTRAL INFORMATION

(71) Applicant: YISSUM RESEARCH DEVELOPMENT COMPANY OF THE HEBREW UNIVERSITY OF JERUSALEM LTD., Jerusalem (IL)

(72) Inventors: Uriel LEVY, Kiriat Ono (IL); Eitan EDREI, Jerusalem (IL)

(21) Appl. No.: 18/558,603

(22) PCT Filed: Jul. 5, 2022
(86) PCT No.: PCT/IL2022/050719

§ 371 Date: Nov. 2, 2023

Related U.S. Application Data
(60) Provisional application No. 63/184,898, filed on May 6, 2021.
(60) Provisional application No. 63/263,462, filed on Nov. 3, 2021.

(51) Int. Cl. G01J 3/26; G01J 3/18; G01N 21/17; G01N 21/64

(52) U.S. Cl. CPC G01J 3/26; G01J 3/1804; G01N 21/6456; G01J 2003/1876; G01N 2021/1772; G01N 2021/1787

(57) ABSTRACT

The disclosure relates to the technique, including systems and methods, for use in optical topographical and/or tomographic 3D imaging of a sample. The system may include (a) a lens unit, chromatically dispersive so that its focal length varies depending on a light wavelength, the lens unit being configured to pass therethrough polychromatic light arriving from and originated at a sample, while selectively collimating those spectral components of the polychromatic light which are in focus based on their wavelengths and origins; and (b) an etalon structure accommodated in an optical path of light being output from the lens unit to receive the collimated light, said etalon structure being configured to operate with multiple resonant wavelengths and to provide respective spectral transmittance peaks at said resonant wavelengths.

### TECHNOLOGICAL FIELD

This disclosure is related to the field of three-dimensional (3D) imaging of objects.

### BACKGROUND ART

References considered to be relevant as background to the presently disclosed subject matter are listed below:
1. York, A. G.; Parekh, S. H.; Nogare, D. D.; Fischer, R. S.; Temprine, K.; Mione, M.; Chitnis, A. B.; Combs, C. A.; Shroff, H., Resolution doubling in live, multicellular organisms via multifocal structured illumination microscopy. Nature Methods 2012, 9 (7), 749-U167.
2. Edrei, E.; Scarcelli, G., Optical focusing beyond the diffraction limit via vortex-assisted transient microlenses. ACS Photonics 2020, 7 (4), 914-918.
3. Lerman, G. M.; Yanai, A.; Levy, U., Demonstration of nanofocusing by the use of plasmonic lens illuminated with radially polarized light. Nano letters 2009, 9 (5), 2139-2143.
4. Horton, N. G.; Wang, K.; Kobat, D.; Clark, C. G.; Wise, F. W.; Schaffer, C. B.; Xu, C., In vivo three-photon microscopy of subcortical structures within an intact mouse brain. Nature Photonics 2013, 7 (3), 205-209.
5. Badon, A.; Bensussen, S.; Gritton, H. J.; Awal, M. R.; Gabel, C. V.; Han, X.; Mertz, J., Video-rate large-scale imaging with Multi-Z confocal microscopy. Optica 2019, 6 (4), 389-395.
6. Wan, Y.; McDole, K.; Keller, P. J., Light-sheet microscopy and its potential for understanding developmental processes. Annual review of cell and developmental biology 2019, 35, 655-681.
7. Zhang, Q.; Pan, D.; Ji, N., High-resolution in vivo optical-sectioning widefield microendoscopy. Optica 2020, 7 (10), 1287-1290.
8. Prevedel, R.; Yoon, Y.-G.; Hoffmann, M.; Pak, N.; Wetzstein, G.; Kato, S.; Schrödel, T.; Raskar, R.; Zimmer, M.; Boyden, E. S., Simultaneous whole-animal 3D imaging of neuronal activity using light-field microscopy. Nature methods 2014, 11 (7), 727-730.
9. Pepe, F. V.; Di Lena, F.; Mazzilli, A.; Edrei, E.; Garuccio, A.; Scarcelli, G.; D'Angelo, M., Diffraction-Limited Plenoptic Imaging with Correlated Light. Physical Review Letters 2017, 119 (24).
10. Beaulieu, D. R.; Davison, I. G.; Kilig, K.; Bifano, T. G.; Mertz, J., Simultaneous multiplane imaging with reverberation two-photon microscopy. Nature methods 2020, 17 (3), 283-286.
   11. Antipa, N.; Kuo, G.; Heckel, R.; Mildenhall, B.; Bostan, E.; Ng, R.; Waller, L., DiffuserCam: lensless single-exposure 3D imaging. Optica 2018, 5 (1), 1-9.
12. Mertz, J., Strategies for volumetric imaging with a fluorescence microscope. Optica 2019, 6 (10), 1261-1268.
13. Dardikman-Yoffe, G.; Mirsky, S. K.; Barnea, I.; Shaked, N. T., High-resolution 4-D acquisition of freely swimming human sperm cells without staining. Science advances 2020, 6 (15), eaay7619.
14. Fan, J. L.; Rivera, J. A.; Sun, W.; Peterson, J.; Haeberle, H.; Rubin, S.; Ji, N., High-speed volumetric two-photon fluorescence imaging of neurovascular dynamics. Nature communications 2020, 11 (1), 1-12.
15. Redding, B.; Liew, S. F.; Bromberg, Y.; Sarma, R.; Cao, H., Evanescently coupled multimode spiral spectrometer. Optica 2016, 3 (9), 956-962.
16. Aieta, F.; Kats, M. A.; Genevet, P.; Capasso, F., Multiwavelength achromatic metasurfaces by dispersive phase compensation. Science 2015, 347 (6228), 1342-1345.
17. Engelberg, J.; Zhou, C.; Mazurski, N.; Bar-David, J.; Kristensen, A.; Levy, U., Near-IR wide-field-of-view Huygens metalens for outdoor imaging applications. Nanophotonics 2020, 9 (2), 361-370.
18. Khorasaninejad, M.; Capasso, F., Metalenses: Versatile multifunctional photonic components. Science 2017, 358 (6367).
19. Cu-Nguyen, PH., Grewe, A., Feβer, P. et al. An imaging spectrometer employing tunable hyperchromatic microlenses. Light Sci Appl 5, e16058 (2016);
20. Dobson SL, Sun PC, Fainman Y. Diffractive lenses for chromatic confocal imaging. Appl Opt 1997; 36: 4744-4748.;
21. Papastathopoulos E, Körner K, OstenW. Chromatic confocal spectral interferometry. Appl Opt 2006; 45: 8244-8252; and
22. Hillenbrand M, Mitschunas B, Brill F, Grewe A, Sinzinger S. Spectral characteristics of chromatic confocal imaging systems. Appl Opt 2014; 53: 7634-7642.

### BACKGROUND

3D imaging applications can be classified into three main categories depending on the expected distance between the imaging system and the imaged objects or expected depth variation of the surface of the imaged object. For example, in the short-range 3D imaging such a distance or the depth variation may be between 0.01 and 1000 microns, and the desired precision may be about 10 nm. In many cases, surface quality check in electronics production and 3D imaging of dynamic biological processes has to be short-range. Useful techniques for short-range 3D imaging include optical profilometry, confocal microscopy, and triangulation. In the mid-range 3D imaging, the distance or the depth variation may be between 10 and 300 cm, and the desired precision may be 1 mm. Such 3D imaging may be needed in 3D printing, face recognition, and augmented reality applications. Useful techniques for the mid-range 3D imaging include structured light techniques and time-of-flight (ToF) techniques. In the long-range 3D imaging, the distance or the depth variation may be between 10 and 300 meters, and the desired precision may be 10 cm. For example, such imaging is useful for autonomous vehicles. Useful techniques for the long-range 3D imaging include time-of-flight (ToF) techniques.

With regard to the short-range 3D imaging, optical microscopy has experienced a renaissance in the past decade greatly stimulated by the introduction of super-resolution modalities. Localization microscopy and structural illumination microscopy are nowadays widely available providing nanoscale lateral resolution, while other techniques based on innovative material structuring are constantly being developed. At the same time, in many cases the depth information is of great interest and requires an additional scan over the axial dimension.

Also, more generally, volumetric imaging with high spatiotemporal resolution is of utmost importance for various applications ranging from aerospace defense and 3D printers to real time imaging of dynamic biological processes.

Thus, obtaining the depth information by an additional scan over the axial dimension leads to an unfortunate compromise between spatial and temporal resolution; indeed, when a large volume is of interest one needs to either sample it with high spatial resolution at the expenses the of temporal resolution or vice versa. Such a tradeoff is often intolerable and renders the modality unsuitable for many applications such as LiDAR or developmental biology where dynamic three-dimensional scenes are of interest.

In particular, three-dimensional sectioning at high spatiotemporal resolution remains the Achilles heel of optical imaging due to the lack of combined axial sub-micron resolution together with a rapid acquisition time. In this context, laser scanning confocal microscopy (LSCM) and multiphoton microscopy (MPM) have been the primary tools for decades to enable three-dimensional imaging with axial resolution up to ˜2 μm. Nevertheless, the time-consuming physical scan over all three dimensions as well as the high energy required to operate MPM has rendered these tools unsuitable for real-time volumetric imaging often required for biological dynamic studies. Recently there have been several intriguing attempts to overcome the aforementioned barrier by using confocal configurations and introducing a series of reflecting pinholes conjugated to different sample plans, or by using chromatic dispersion to obtain axial sectioning. Yet, due to the inherent confocal mechanism, a careful adjustment of the pinholes is required to meet the conjugation demand which limits the number of planes imaged simultaneously and necessitates a cumbersome alignment procedure.

By selectively illuminating a single plane at a time, light sheet microscopy and the more recent HiLO microscopy present a significant speed advantage. However, they require high optical clarity of the sample and typically achieve a lower axial resolution. Optical coherence tomography (OCT) utilizes low coherence sources to enable axial sectioning and is the gold-standard tool for retinal imaging, yet, both lateral and axial resolution are compromised to support a significant depth range thus rarely exceeding ˜5 μm. The quest for volumetric microscopic imaging techniques stimulated a variety of new ideas; light-field advanced modalities, reverberation microscopy or diffuser assisted computational reconstruction where recently demonstrated. These emerging strategies often come with a fundamental trade-off between axial and lateral resolution or require some a-priori knowledge of the sample.

### GENERAL DESCRIPTION

There is a need for a convenient technique for obtaining instantaneous depth information to facilitate real-time volumetric imaging (a) while still using scanning in lateral dimensions or (b) by performing the volumetric imaging of a full scene or of its substantial part in a single shot.

The systems and methods are suited for various applications such as object scanning for 3D printers (e.g. to fabricate a prosthesis or prepare a complementary surface, as in tooth crown), autonomous automotive sensing as well as microscopic and macroscopic topography and tomography. For example, applications include surface inspection in electronics production, for instance, for ascertaining its quality; as well as 3D metrology applications in wafer packaging; inspection of conductive vertical connections, e.g. bumps and pillars made of gold or copper; and inspection of bumps made of other materials, e.g. solder. Further applications include 3D imaging of dynamic biological processes.

To enable both tasks (a) and (b) above, that is for imaging of all three dimensions without the need to physically scan with the imaging system the axial/depth axis (by moving or tuning the system or its part, or by moving a sample), the present disclosure presents a technique named spectral gating (SG) or spectrally gated microscopy (SGM) when applied to micro-scaled objects.

The concept of SG is based on two main components: 1. A chromatically dispersive lens unit, for example a flat optical lens (i.e. diffractive or metalens) with a different focal length for each wavelength (for example, in a certain desired region around a nominal wavelength); 2. An optical dispersion device such as an etalon structure accommodated in an optical path of light being output from the lens unit for receiving the collimated light. The etalon structure is to operate with multiple resonant wavelengths, and is to provide a respective spectral transmittance peak at each of said resonant wavelengths.

For example, the etalon structure may be a Fabry-Perot etalon (FP)—in some embodiments this can be replaced by a simpler or more complex component. Some embodiments with such an etalon enable three-dimensional acquisition at a single shot, as explained later.

A system combining at least the two components as above may be used in 3D imaging of a sample. The chromatically dispersive lens unit will allow passing therethrough of polychromatic light arriving from and originated at the sample, i.e. emerging from the sample, as well as possibly light arriving from random sources (for example, light scattered on dust in the air). However, the lens unit will selectively collimate those spectral components of the polychromatic light which are in focus based on their wavelengths and origins (where the selective collimation means that the lens will not collimate other spectral components, i.e. those which are not in focus, depending on their wavelengths and origins). The uncollimated spectral components will be attenuated in a consequential output of the etalon, to a degree depending on how far they are from the collimation. Also, further attenuation will be experienced by those spectral components, which are further from a resonant wavelength corresponding to a distance from the lens to this location. However, this further attenuation (for equally collimated or uncollimated components) will be much greater for those components, which are further from any resonant wavelength of the etalon structure. Overall, depending on the task (a) or (b) and other aims with regard to the field of view, contrast, allowable aberrations etc, the system will provide a consequential output of the etalon structure, in which presence of one or more of resonant wavelengths will be indicative of that the respective one or more spectral components have originated from a correspondingly distanced location at the sample.

Herein, if for example many spectral components succeed in passing from the location at the sample through the lens unit and the etalon structure, their intensities should in average decline from a maximal intensity which can be expected to be reached somewhere approximately in a range between a wavelength of a spectral component collimated the best by the lens unit and its closest resonant wavelength of the etalon structure (in some cases, when resonant wavelengths start to substantially depend on variations of the incidence angle, the closest resonant wavelength would shift depending on the location at the sample). Thus, the consequential output of the resonant structure may present a number of spectral components with different intensities, but having a peaking or a bell-like envelope, as in FIG. 7(a) described below.

In the 1st aspect of the present disclosure, there is provided a system for use in optical topographical and/or tomographic 3D imaging of a sample, comprising:
a lens unit, chromatically dispersive so that its focal length varies depending on a light wavelength, said lens unit being configured to pass therethrough polychromatic light arriving from and originated at a sample, while selectively collimating those spectral components of the polychromatic light which are in focus based on their wavelengths and origins, and
an etalon structure accommodated in an optical path of light being output from the lens unit to receive the collimated light, said etalon structure being configured to operate with multiple resonant wavelengths and to provide respective spectral transmittance peaks at said resonant wavelengths.

In the 2nd aspect of the present disclosure, there is provided the system as in the 1st aspect, wherein said etalon structure is configured to provide simultaneous operation of said multiple resonant wavelengths.

In the 3rd aspect of the present disclosure, there is provided the system as in the 1st aspect, wherein said etalon structure is tunable to operate with different resonance conditions each characterized by one of said multiple resonant wavelengths.

In the 4th aspect of the present disclosure, there is provided the system as in any one of the preceding aspects, wherein the lens unit comprises a dispersive flat optical lens.

In the 5th aspect of the present disclosure, there is provided the system as in the 4th aspect, wherein the dispersive flat optical lens is a diffractive lens.

In the 6th aspect of the present disclosure, there is provided the system as in the 4th or 5th aspect, wherein the dispersive flat optical lens is a meta-lens.

In the 7th aspect of the present disclosure, there is provided the system as in any one of preceding aspects, wherein the lens unit comprises a diffractive zone plate and a refractive lens.

In the 8th aspect of the present disclosure, there is provided the system as in any one of preceding aspects, wherein the lens unit at a nominal wavelength has a focal length in the range of 100 μm (microns) to 1 m.

In the 9th aspect of the present disclosure, there is provided the system as in any one of preceding aspects, further comprising an optical detector configured to detect an output of the etalon structure consequential to said polychromatic light arriving from and originated at the sample and generate measured data indicative thereof.

In the 10th aspect of the present disclosure, there is provided the system as in the 9th aspect, wherein said optical detector comprises a spectrophotometer.

In the 11th aspect of the present disclosure, there is provided the system as in the 9th or 10^{th} aspect, wherein the optical detector comprises an image sensor comprising a CCD image sensor or an active-pixel sensor.

In the 12th aspect of the present disclosure, there is provided the system of any one of the 9^{th} to 11^{th} aspect, wherein said optical detector comprises a multispectral camera, optionally configured to operate with from 3 up to 30 spectral bands or other spectral modalities in each pixel, or a hyperspectral camera, optionally configured to operate with 30 to 200, or more, spectral bands or other spectral modalities in each pixel.

In the 13th aspect of the present disclosure, there is provided the system as in the 12th aspect, wherein the spectral bands are distributed contiguously.

In the 14th aspect of the present disclosure, there is provided the system as in the 13th aspect, wherein said other spectral modalities are modalities of time-domain Fourier transform imaging.

In the 15th aspect of the present disclosure, there is provided the system as in any one of the 12th to 14th aspect, wherein a free spectral range of the etalon structure is larger than a spectral resolution provided by the multispectral or hyperspectral camera.

In the 16th aspect of the present disclosure, there is provided the system as in any one of the 12th to 15th aspect, wherein the lens unit is adapted to provide a longitudinally chromatic aberration so that the focal length changes in a spectrum detectable by the multispectral or hyperspectral camera by at least 1%, or 3%, or 10% of a nominal focal length.

In the 17th aspect of the present disclosure, there is provided the system of as in any one of preceding aspects, wherein said optical photodetector is configured to detect light with at least one wavelength being in a range from 300 nm to 1 mm.

In the 18th aspect of the present disclosure, there is provided the system as in any one of the 9th to 17th aspects, further comprising a control unit configured and operable to process the measured data and calculate a distance from the lens to a location at the sample, based on a spectral signal from said optical detector.

In the 19th aspect of the present disclosure, there is provided the system as in any one of 11th to 17th aspects, further comprising a control unit configured and operable to process the measured data and calculate a distance from the lens unit to a location at the sample based on a spectral signal from any one of the pixels of the image sensor of the optical detector.

In the 20th aspect of the present disclosure, there is provided the system as in any one of the 18th and 19th aspects, wherein the control unit is configured to calculate the distance by taking into account also a spectral profile of light illuminating the sample.

In the 21th aspect of the present disclosure, there is provided the system as in any one of the 18th and 20th aspects, wherein the control unit is configured to calculate the distance based on a wavelength of an only one spectral band from spectral bands of the detector when the spectral signal represents a detection by said only one spectral band of a part of the polychromatic light originating at the location at the sample.

In the 22nd aspect of the present disclosure, there is provided the system as in any one of the 18th and 21th aspects, wherein the control unit is configured to calculate the distance based on a wavelength of an only one spectral band from spectral bands of the detector when the spectral signal represents a detection by two spectral bands from the spectral bands of the detector of a part of the polychromatic light originating at the location at the sample, wherein the calculation is based on that spectral band which produced a relatively greater signal.

In the 23th aspect of the present disclosure, there is provided the system as in any one of the 18th and 22nd aspects, wherein the control unit is configured to calculate the distance by estimating a central wavelength of an envelope of a spectral distribution of a part of the polychromatic light originating at the location at the sample, when the spectral signal represents a detection by three or more of spectral bands of the detector of said part.

In the 24th aspect of the present disclosure, there is provided the system as in any one of the 9th to 23rd aspects, further comprising at least one of (a) an achromatic imaging lens system for directing that light output of the etalon structure which is to arrive to the optical detector, and (b) a reference arm optical detector and a reference arm achromatic imaging lens system for focusing a part of light reflected and/or emitted by the sample on the reference arm optical detector while bypassing the etalon structure.

In the 25th aspect of the present disclosure, there is provided the system as in any one of the preceding aspects, wherein the etalon structure is a Fabry-Perot etalon.

In the 26th aspect of the present disclosure, there is provided the system as in any one of the preceding aspects, wherein a finesse of the etalon structure is in a range of from 10 to 150, or from 15 to 100, or from 25 to 75.

In the 27th aspect of the present disclosure, there is provided the system as in any one of the preceding aspects, wherein a free spectral range of the etalon structure is in a range of 10 nm-0.001 nm, or 10 nm-0.01 nm, or 10 nm-0.1 nm.

In the 28th aspect of the present disclosure, there is provided the system as in any one of the preceding aspects, wherein the lens unit is configured to collect the polychromatic light from a field of view comprising angles of arrival up to 30°, or 10°, or 5° measured from an optical axis of the system.

In the 29th aspect of the present disclosure, there is provided the system as in any one of the preceding aspects, further comprising a source of polychromatic illuminating light configured to illuminate a region at the sample to produce at least a part of the polychromatic light originating at the sample as a first or other order reflection and/or scattering and/or fluorescent and/or other response from an external surface and/or an internal surface and/or one or more depths of the sample.

In the 30th aspect of the present disclosure, there is provided the system as in the 29th aspect, wherein the source of polychromatic illuminating light is adapted to provide broadband illumination comprising spectral components corresponding to a plurality of the resonant wavelengths of the etalon structure.

In the 31st aspect of the present disclosure, there is provided the system as in the 29th aspect, wherein the source of polychromatic illuminating light is adapted to provide illumination with a spectral intensity distribution peaking at a plurality of the resonant wavelengths of the etalon structure.

In the 32nd aspect of the present disclosure, there is provided the system as in any one of the 29th and 31st aspects, further comprising a machine-readable memory or memory carrier storing a record on a predetermined or measured spectral profile of the polychromatic illuminating light for determining a spectral profile of light illuminating the sample.

In the 33rd aspect of the present disclosure, there is provided the system as in any one of the 29th to 32nd, comprising at least one polarizing unit, accommodated in an optical path of the illuminating light to the sample and/or in an optical path of the collimated light to the etalon structure.

In the 34th aspect of the present disclosure, there is provided the system as in the 33rd aspect, wherein the at least one polarizing unit is configured to provide to light passing therethrough a TE-mode, or a TM mode, or a circular polarization.

In the 35th aspect of the present disclosure, there is provided the system as in any one of the preceding aspects, further comprising an optical splitter accommodated in an optical path of light output from the etalon structure and configured to split from it the consequential output of the etalon structure.

In the 36th aspect of the present disclosure, there is provided the system as in any one of the preceding aspects, wherein the dispersive lens unit is configured to have a longitudinally chromatic aberration satisfying at least one from an inequality

$$\vert \frac{Δλ}{λ} \vert < 0.01 \vert \frac{Δ f}{f} \vert ,$$

an inequality

$$\vert \frac{Δ λ}{λ} \vert < 0.1 \vert \frac{Δ f}{f} \vert ,$$

an inequality

$$\vert \frac{Δ λ}{λ} \vert < 0.5 \vert \frac{Δ f}{f} \vert$$

and approximately an equlity

$$\vert \frac{Δ λ}{λ} \vert = \vert \frac{Δ f}{f} \vert ,$$

for two adjacent resonant wavelengths of the etalon structure at a normal incidence angle separated by a wavelength difference of Δλ, where the interval of Δλ between the two adjacent resonant wavelengths covers a nominal wavelength λ of the system, Δf is a difference between focal lengths of the dispersive lens unit at the two adjacent resonant wavelengths, and f is a focal length of the dispersive lens unit at the nominal wavelength of the system.

In the 37th aspect of the present disclosure, there is provided the system as in any one of the preceding aspects, wherein the etalon structure is tunable for adapting the resonant wavelengths thereof to a range of depths of the sample.

In the 38th aspect of the present disclosure, there is provided the system as in any one of the preceding aspects, wherein the lens unit comprises an array of dispersive flat optical lenses.

In the 39th aspect of the present disclosure, there is provided the system as in any one of the preceding aspects, wherein the etalon structure is configured with the multiple resonant wavelengths respectively varying for a range of incidence angles of collimated light on the etalon structure.

In the 40th aspect of the present disclosure, there is provided the system as in any one of the preceding aspects, wherein the etalon structure is configured with the spectral transmittance peaks respectively varying for a range of incidence angles of collimated light on the etalon structure.

In the 41st aspect of the present disclosure, there is provided the system as in any one of the preceding aspects, comprising a spectrometer accommodated to detect a spectral distribution of said consequential output of the etalon structure.

In the 42nd aspect of the present disclosure, there is provided the system as in any one of the preceding aspects, further comprising a support stage for supporting a sample under measurements, the system being configured and operable to affect a relative displacement in at least one lateral dimension between said stage and an optical unit formed by the dispersive flat lens unit and the etalon structure.

In the 43rd aspect of the present disclosure, there is provided an optical unit for use in a microscope, the optical unit comprising the system of any one of the 1st to 41^{st} aspects.

In the 44th aspect of the present disclosure, there is provided a method for use in optical topographical and/or tomographic 3D imaging of a sample, comprising:
passing through a lens unit, chromatically dispersive so that its focal length varies depending on a light wavelength, polychromatic light arriving from and originated at a sample, while selectively collimating those spectral components of the polychromatic light which are in focus based on their wavelengths and origins, and
receiving the collimated light at an etalon structure, accommodated in an optical path of light being output from the lens unit and configured to operate with multiple resonant wavelengths to provide respective spectral transmittance peaks at said resonant wavelengths to the collimated light.

In the 45th aspect of the present disclosure, there is provided the method as in the preceding aspect, comprising passing a part of the collimated light though the etalon structure.

In the 46th aspect of the present disclosure, there is provided the method as in the 44th or 45th aspect, further comprising detecting an output of the etalon structure consequential to said polychromatic light arriving from and originated at the sample, and generating measured data indicative of said output, with an optical detector.

In the 47th aspect of the present disclosure, there is provided the method as in the 46th aspect, further comprising processing with a control unit the measured data to calculate a distance from the lens to a location at the sample, based on a spectral signal from said optical detector.

In the 48th aspect of the present disclosure, there is provided a non-transitory machine-readable medium storing instructions executable by a processor, the non-transitory machine-readable medium comprising:
instructions to calculate with the measured data generated by the method of the 46^{th} aspect a distance from the lens to a location at the sample, based on a spectral signal from said optical detector.

In view of the above, this disclosure also encompasses in particular the following embodiments, which for convenience are discussed in the following description before the above aspects:
1. SGM—tomographic images via spectral gating using, for example, flat optics and a FP. This technique is well suited for very high-resolution measurements, yet, requires a lateral scan and is more expensive.
2. SGT—topographic images. (If the topography measure of a surface is desired, then at times the etalon, such as FP, can be omitted as reflections from various planes are not expected. The spectrometer can be replaced by an RGB camera which enables full field 3D acquisition with many advantages over some current technologies, as above).
3. Full-field SGM—a single shot acquisition of the three-dimensional scene—topography and tomography.

### BRIEF DESCRIPTION OF THE DRAWINGS

In order to better understand the subject matter that is disclosed herein and to exemplify how it may be carried out in practice, embodiments will now be described, by way of non-limiting examples only, with reference to the accompanying drawings:

FIGS. 1a-d: (a) Schematic illustration of the SGM mechanism with monochromatic light. (b) Schematic illustration of the SGM mechanism with several point sources located at various axial locations. (c) Optical diagram showing the ray propagation from an out-of-focus location. (d) A simulated three-dimensional PSF of the system presented in (a) without (left) and with (right) the FP etalon.

FIGS. 2a-f: (a) Setup describing the SGT method. (b) Ray tracing of a diffractive lens. (c) Grid object (R, G, B=1). (d) Grid image when placed far away. (e) Grid image when placed at 1.35 meters. (f) Grid image when placed at 65 cm.

FIG. 3: Schematic illustration of the SGM concept when operated in a full-field configuration. Reflections from every location in space are encoded by the reflected spectrum which is recorded by the hyperspectral camera. Parallel rays from the output of the lens unit (or more generally, light emerging from locations which are approximately in-focus) are transmitted with much greater efficiency at about resonant wavelengths, and therefore due to the varying focal plane of the meta/diffractive lens they generate a peak on the recorded spectrum (which resolution allows detecting the envelope, but not necessarily individual resonant wavelengths).

FIGS. 4a-e: (a) Optical setup for SGM quantification. (b) An axial PSF showing the sectioning capability of the system. (c) An axial scan of a sample containing a thin silicon nitride layer deposited on top of a silicon substrate. (d) Axial resolution for a constant NA (=0.85) at various focal lengths. (e) Axial resolution for a constant focal length (f=4.3 mm) at various NA values.

FIGS. 5a-f: (a) A partial SEM image of the meta lens. (b) An enlarged region marked by a red dashed line in (a). (c) The PSF of the meta-lens, scale bar=500 nm. (d) The experimental MTS (blue dots) compared to the diffraction limited MTF (black dashed line) obtained by Virtuallab Fusion. (e) Schematics of the spectral focusing dispersion generated by the meta-lens. (f) Axial sectioning of different planes is obtained by wavelength variation.

FIGS. 6a-e: (a) Single shot axial acquisition setup. (b) A Danish krone (scale bar=1 mm). (c) The spectrums obtained from the selected locations in (b). (d) A tomographic image of the heart region. (e) A USA dime coin (scale bar=1 mm) and the corresponding tomographic image of a selected region.

FIGS. 7a-b: (a) The transmission spectrum through the Fabry-Perot of light reflected off a surface (illuminated with a broad illumination). The peak of the envelope function indicates the axial location from which the reflection arrived. (b) Same transmission spectrum as in (a) where the X-axis has been transformed to axial location.

FIGS. 8a and 8b schematically illustrate a system for use in optical topographical and/or tomographic 3D imaging in accordance with the present disclosure.

FIGS. 9a and 9b present examples of the chromatically dispersive lens unit according the present disclosure.

FIGS. 10a-c present artistic cross-sectional sketches of various lens types. FIG. 10(a) relates to a refractive lens, FIG. 10(b) relates to a diffractive lens with flattening due to the division into radial zones, and FIG. 10(c) relates to a metalens showing nanoantennas for phase control.

FIG. 11: schematically illustrates a variation of the system for use in optical topographical and/or tomographic 3D imaging in accordance with the present disclosure.

FIG. 12 presents an example scheme of a method which can be used for optical topographical and/or tomographic 3D imaging of a sample.

FIG. 13 schematically shows a non-transitory machine-readable medium storing instructions executable by a processor, according to the present disclosure.

FIG. 14a-b: show (a) A schematic ray propagation diagram through a lens. (b) A schematic transmission diagram through a FP.

FIG. 15a-f relate to two examples of the lens designed for the 3D vision by the inventors: (a)-(c) show PSF, MTF and chromatic focal length shift respectively for one lens, and (d)-(f) show the same for the other lens.

FIG. 16 shows examples of the transmission spectra through the Rubidium vapor cell (black dashed line) and through the FP (blue line).

FIG. 17 presents an illustration on a relationship between the relative measured thickness (blue dots) and the corrected measured thickness obtained after multiplication by the refractive index (red dot): in case of tomography the depth measured by all optical modalities is affected by the refractive index of the interrogated layer of the specimen.

FIGS. 18a-b relate to metalense design and show (a) Phase map at varying pillar heights (x axis) and radii (y axis). (b) A plot of the dashed line in (a), i.e. at a height of 1100 nm.

### DETAILED DESCRIPTION OF THE ASPECTS AND EMBODIMENTS

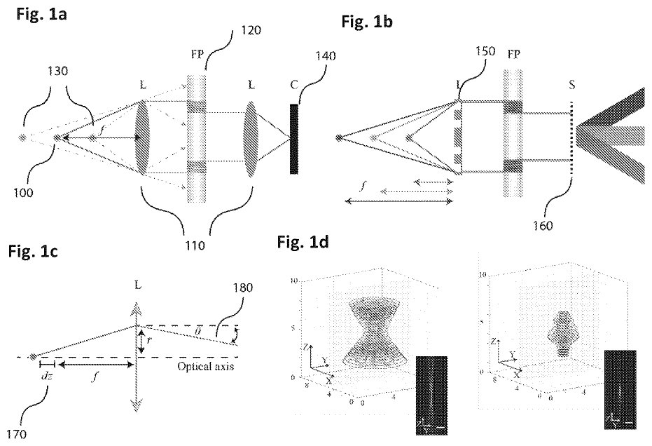

Reference is made to FIGS. 1a-d schematically exemplifying the main functional elements used by the inventors. FIGS. 1a-b illustrate schematically the SGM mechanism with monochromatic light, and optical diagram showing the ray propagation from an out-of-focus location, emphasizing also the role of the etalon useful for understanding the origin of the principle of SG.

In this example, the system encompasses a monochromatic light source (100), an imaging system (110) including, for the sake of simplicity, in this case two achromatic refractive lenses L and Fabry-Perot etalon (120), in this case of a high finesse. Light emitted by the source when located at the focal spot of the first lens will exit the back aperture as a collimated beam. Hence, by tuning the FP resonance the full transmission of said emission can be ensured. However, any additional light emerging from a different plane (130) rather than the focal plane corresponding to the wavelength of the monochromatic light source, will exit the back aperture of the lens as a converging or diverging beam. Therefore, it will not comply with the pre-tuned resonance of the high-finesse etalon structure, and will be attenuated or rather even rejected by the etalon.

That is, depending on aims of the imaging (and pre-tuning of the etalon), the SG mechanism can be used to ensure that only or mostly a corresponding spectral component of the light originating from some focal plane or spot located at the sample arrives at the detector/camera (140). This is accomplished via a combination of the spectral and simultaneously angular attenuation/rejection of other spectral components originating from this focal plane or spot; as well as similar angular attenuation/rejection of most of the light emerging from other planes or spots. Such a spectral-angular attenuation/rejection is in contrast to the spatial rejection used in confocal configurations.

Hence, the SG mechanism can be used to enable single shot acquisition of the entire axial axis by introducing polychromatic light. In such a case, the chromatically dispersive lens unit including, for example, a flat optical component (e.g. a diffractive lens, a metalens, and/or a diffractive zone plane combined with a refractive lens) will support many focal planes. The principle of single shot SG using, for example, a metalens is schematically presented in FIG. 1b; the multiple focal planes provided by the metalens (150) enable the spectral decoding of information from different planes. Hence, by separating and analyzing each wavelength individually using, as an example, a spectrometer (160), sectioning of different planes is achieved simultaneously without the need for depth scanning (for example, with an adjustable confocal aperture).

Consequently, the present disclosure introduces a new concept named hereby spectrally gated microscopy (SGM) which enables a single shot interrogation over the full axial dimension or a substantial portion of it while maintaining a sub-micron sectioning resolution. SGM can utilize two features as above (i.e. the chromatically dispersive lens unit and the etalon structure). When the lens unit is made with, for example, flat optics (i.e. meta and diffractive lenses), this enables—a short focal length and strong chromatic aberrations. Performing three-dimensional imaging of millimeter-scale samples using SGM while scanning only the lateral dimension(s) yields significant benefits. In this regard, the SGM paradigm for three-dimensional sectioning, i.e. the name “spectrally gated microscopy” can be opposed to spatial (LSCM, MPM) or coherent time (OCT) sectioning mechanisms: SGM attenuates/rejects out-of-focus light through a resonance mismatch of spectral components arriving at different angles. The spectral gating mechanism can provide sub-micron axial sectioning, as examples below show, with resolution higher than offered by some of the state-of-the-art technologies, together with a single-shot axial acquisition which eliminates or reduces the need for depth scanning. The very substantial advantage of this modality arises from the transformation of information from the spatial domain to the spectral one, in which chromatic multiplexing can be realized by exploiting the chromatic aberrations, for example of flat optical components such as meta-lenses. The combination of the SGM mechanism with, for example, the meta-lens technology enables parallel multiplane imaging and overcomes the spatiotemporal barrier imposed by the requirement for imaging large volumes at high resolution.

In this regard, in general, only some imaging techniques use a chromatically dispersive lens unit: much more often chromatic aberrations are corrected. Still, for example, in one publication relating to imaging with the chromatic confocal microscope an employed lens unit is called a tunable hyperchromatic lens (HCL) (see Cu-Nguyen, PH., Grewe, A., Feβer, P. et al. An imaging spectrometer employing tunable hyperchromatic microlenses. Light Sci Appl 5, e16058 (2016)). According to this publication: The HCL is a combination of a diffractive lens and a hydraulically-tunable refractive lens. The tunable lens consists of a liquid-filled microfluidic cavity bounded by a distensible polymer membrane, which forms the refractive surface. The diffractive part is designed as a Fresnel lens with a focal length strongly dependent on wavelength. Due to the high dispersion, the diffractive lens is employed to focus different wavelengths at different positions distributed along the optical axis. The description in the above publication relating to such an implementation of the lens unit is incorporated herein by reference.

Further, according to the above publication, three following publications aim at using a diffractive optical zone plate or a combination of refractive elements with large chromatic aberration, for three-dimensional imaging without the axial scanning as required in conventional confocal microscopes: Dobson SL, Sun PC, Fainman Y. Diffractive lenses for chromatic confocal imaging. Appl Opt 1997; 36: 4744-4748.; Papastathopoulos E, Körner K, Osten W. Chromatic confocal spectral interferometry. Appl Opt 2006; 45: 8244-8252; and Hillenbrand M, Mitschunas B, Brill F, Grewe A, Sinzinger S. Spectral characteristics of chromatic confocal imaging systems. Appl Opt 2014; 53: 7634-7642.

The confocal microscopes use a pinhole at the back to perform 3D imaging. Due to this pinhole there is the need to scan along lateral dimensions (X and Y). Hence, to facilitate three-dimensional sectioning, there are technologies which rely on mechanisms to reject light from adjacent out-of-focus planes either spatially or by other means; yet, the combination of rapid acquisition time and high axial resolution is still elusive.

Similarly, in the above embodiment of FIG. 1b the lateral dimension(s) still needs to be scanned. However, there is no limitation of the pinhole and, according to the present disclosure, a combination of the chromatically dispersive lens unit, presented in this case by meta-lens 150, and the etalon structure, presented in this case by the Fabry-Perot etalon, makes it possible to operate in various configurations such that the entire three-dimensional scene would captured simultaneously.

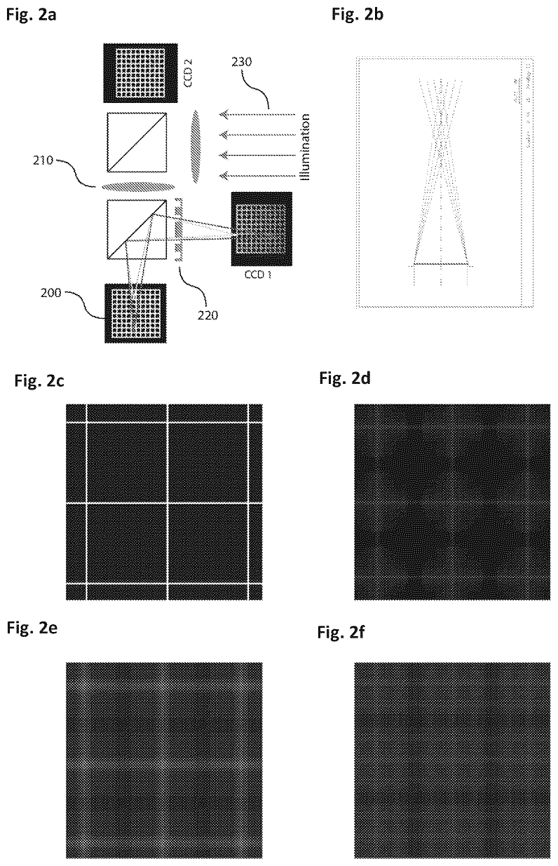

In particular, one such an embodiment is called hereby spectral gating topography (SGT). SGT works well when the topography of the object is of interest and is described in FIG. 2a (showing ray tracking of a diffraction lens in an SGT setup), even lacking in this case optional etalon structure between the chromatically dispersive lens unit (implemented by metalens 220) and a photodetector (implemented by CCD 1). That is, the scene (200), e.g. a sample, is imaged using two imaging systems, among which one is chromatically corrected (210), i.e. focusing all wavelengths to the same plane, and the other is a chromatically dispersive lens system, in this case a flat optical uncorrected system (220). Assuming for example a polychromatic grid object, the flat optical system will show a different color depending on the axial location of the grid; if for instance at a point ‘P’ of the object the “green” wavelength is at focus, all other wavelengths will be defocused and the RGB values of the CCD will vary correspondingly at location ‘P’ (high G value vs low R,B values). The RGB value at each pixel can be translated into depth if the specific parameters of the flat optical lens are known. Since the spectrum of the object is generally unknown (i.e. there is no justified assumption of a uniform white emission), the corrected arm will provide the true RGB values at each location of the object and the uncorrected system will provide the amount of shift in these values which can be correlated to the depth value at the different locations. In this embodiment the object can be either passively imaged using the natural reflected light, or alternatively, a grid or doted pattern (230) can be projected on the scene (200) and the system will analyze the spectral variations of the reflected light. To reduce cost of SGT only three values of the spectrum are known (RGB) instead of a spectrometer assigned to each pixel, however, it is possible to obtain high spectral accuracy by fitting the values to a gaussian-like envelope and tracking its' shifts.

On the macro scale, to measure the 3D topography of objects, some state-of-the-art technologies apply structured light illumination and extract depth information based on patterning variations after many frames are acquired. In this context the SGT technology presented in this disclosure offers advantages from several aspects: 1. Only a single frame of the scene is required for each viewing point. 2. The system can become compact as there is no need for a large angular separation between the projection and the detection arms. 3. Since the spatial variations are not of interest, it is much easier to handle various types of surfaces where reflection is ambiguous in terms of spatial distribution. 4. Data processing analysis is dramatically simplified as only the RGB values are needed, and this reduces both power consumption and processing strength (or complexity) requirements. However, it should be noted that these advantages are greatly enhanced when the etalon structure as presented above or elsewhere in this disclosure is accommodated between the chromatically dispersive lens unit and the photodetector.

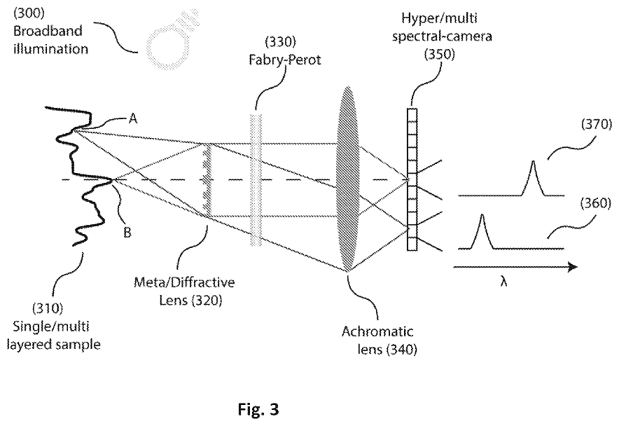

Another embodiment, full field SGM, in which a three-dimensional tomographic scene or at least a substantial volumetric portion of it, is captured within a single shot, is obtained as described with reference to FIG. 3. A broadband illumination (300) from a source of polychromatic illuminating light with a known pre-measured spectrum is applied to the sample of interest (310). The back reflected light is collected using a chromatically dispersive lens unit, for example, a metalens or diffractive lens (320). The collected light is transmitted through an etalon structure, implemented in this example by a Fabry-Perot etalon (330), and focused using an achromatic lens (340) onto a hyperspectral or multispectral camera (350). If the free-spectral-range of the etalon structure, such as the Fabry-Perot, is chosen to be larger than the spectral resolution of the camera, the following outcome will manifest: rays emerging approximately parallel from the back aperture of the lens (either parallel to the optical axis or otherwise) will yield higher intensity than those exiting with many angles.

For instance, consider point ‘A’ in FIG. 3: for the full field SGM, the etalon structure may have a not so high finesse as for the case with the lateral scanning, and it can be approximated that there will be a range of wavelengths for which point ‘A’ is more or less at the focal plane of the lens (for a flat uncorrected lens each single wavelength has a different focal plane, but the lens has a Rayleigh range; thus, when the expression that a range of wavelengths is in focus at the same plane has to be interpreted remembering about this range). Within this range there may be several resonances of the FP, because the FSR may be set smaller than this range. This range will exit the back aperture parallel (blue lines on FIG. 3). Within this relatively broad range, some will also coincide with the resonances of the Fabry-Perot etalon and will be efficiently transmitted. Any wavelength for which point ‘A’ is not at the focal plane of the lens, will generate a converging/diverging distribution of rays after the lens. Among these rays some might be circumstantially transmitted through the Fabry-Perot, but there is no single-wavelength that will be fully transmitted since for any given wavelength the angular distribution of rays ensures that only a slight portion will match a resonance. Hence, the envelope (360) of the spectrum acquired by the camera conjugated to point ‘A’ will show a peak at approximately the wavelength for which this location is to the most degree at some focal plane of the diffractive lens or metalens 320.

Similarly, for point ‘B’ a different range of wavelengths approximately matches the focal plane of the lens in the sense that this range would pass through the etalon structure (again, with higher transmittances for those wavelengths in the range which are better collimated by the lens unit and closer to resonant wavelength for this point ‘B’), hence, they will yield a strong transmission which will manifest as a series of resonances with spectrally peaking envelope centered at a different wavelength (370). This analysis is also valid for multilayered samples; the spectral signature will contain several peaks corresponding to the various reflection depths.

Hence, the spectrums (360, 370) in FIG. 3 are illustrated only in their envelope parts; it should be remembered that within each spectral envelope peak many peaks arise due to the free-spectral-range of the Fabry-Perot. The envelope width defines the axial resolution of the system, and is determined based on the Finesse of the etalon structure such as Fabry-Perot and the chromatically dispersive lens unit parameters (as discussed later).

To evaluate the axial sectioning resolution which can be provided by SGM, the inventors have considered a point source shifted by dz along the optical axis and away from the focal point (FIG. 1c, 170, for a full derivation see supplementary section 1 in the Appendix). The rays emerging from the point source will exit the back aperture of the lens with an angle:

$$θ = a tan ( \frac{r}{\frac{{f}^{2}}{dz} + f} ) \qquad ( 1 )$$

where r is the distance from the center of the lens to the intersection of the ray with the lens surface, and f is the lens focal length. The characteristics of the FP will determine whether light arriving with angle θ (180) characterizing a deviation from the collimated condition or in other words convergence or divergence, will be attenuated or rejected; the angle θ corresponding to a transmission value T through such etalon structure as the FP is given by:

$$θ = a cos [ \frac{λ}{2 π nD} \cdot [ {( - 1 )}^{m} \cdot a sin ( \sqrt{\frac{1 - T}{4 T / {π}^{2} \cdot {F}^{2}}} ) + m π ] ] \qquad ( 2 )$$

where λ is the incident wavelength, D, n, F are the FP thickness refractive index and finesse respectively. Comparing the right-hand side of equations (1), (2) the axial resolution dz can be expressed as:

$$dz = \frac{{Kf}^{2}}{r - fK} \qquad ( 3 )$$

where K is a constant determined by the FP characteristics:

$$K = tan ( a cos [ \frac{λ}{2 π nD} \cdot [ {( - 1 )}^{m} \cdot a sin ( \sqrt{\frac{1 - T}{4 T / {π}^{2} \cdot {F}^{2}}} ) + m π ] ] ) \qquad ( 4 )$$

In this derivation there are two free parameters, T and r which together determine the contrast demand imposed upon the system. The value of T defines the threshold under which transmission is considered negligible, while r determines the radius at the lens plane from which light is blocked (i.e 0<r<Rₗₑₙₛ). For instance, by setting r=0.32·R where R is the radius of the lens, all photons impinging on the lens aperture beyond the radial location of 0.32·R will be blocked, corresponding to ninety percent of the lens area. Substituting the said value into equation (3) and introducing the numerical aperture (NA) in lieu of R yields:

$$d z = \frac{Kf \cdot \sqrt{1 - N {A}^{2}}}{0.32 \cdot NA - K \cdot \sqrt{1 - N {A}^{2}}} \qquad ( 5 )$$

In order for SGM to enable a multiplane single shot acquisition, the naturally large chromatic aberrations as provided by meta-lenses can be utilized. In the following, it is assumed for the sake of simplicity that the chromatically dispersive lens unit is presented by just one lens, but clearly the overall of the unit focal distance can be tuned by adding further lenses or other focusing optical elements. For a chromatically dispersive (i.e. chromatically uncorrected or uncompensated) diffractive lens or particularly for chromatically dispersive (again, i.e. chromatically uncorrected or uncompensated) metalenses it is well known that the change in focal length approximately relates to the wavelength shift:

$$\frac{Δ λ}{λ} = \frac{Δ f}{f} , \qquad ( 6 )$$

where λ is the designed nominal wavelength (which is determined by the design of the lens, for example the periodicity of the rings for the diffractive lens). From equation (6) the axial field of view can be expressed as:

$$Δ Z = f \frac{Δ λ}{λ} ,$$

hence, for a broadband source, a significant axial range can be covered by focal points of different wavelengths, which can then be spectrally dispersed and analyzed separately. Assuming a high finesse FP, the value of K is very small and equation (3) can be approximated as:

$$d z = \frac{K {f}^{2}}{r}$$

and dividing the two expressions yields:

$$\frac{Δ Z}{dz} = \frac{R \cdot Δλ}{f \cdot K λ} \approx NA \cdot \frac{Δ λ}{K λ} \qquad ( 7 )$$

Equation (7) indicates that the number of sectioned planes contained within the axial field of view for given values of K, A, AA is determined by the NA of the metalens: Higher NA yields more sectioned planes within the axial field of view. The above derivation is geometric in nature. Diffraction effects become significant particularly at high NA values and should be accounted for (see supplementary section 1).

To demonstrate the powerful effect of SGM the inventors have performed a simulation using the Virtuallab Fusion software which offers a full field-propagating tool (FIG. 1c, see methods). The exact scenario of FIG. 1a was simulated, i.e. a monochromatic emitting point source was shifted along the optical axis while the intensity distribution at every location was imaged and recorded by the camera. The specific parameters of the simulation were: f=250 μm, D=6.743 mm, F˜30, NA=0.7. As can be seen in the left panel of FIG. 1d, in the absence of the FP a typical point-spread-function (PSF) is obtained, light is not rejected and therefore the total energy at each plane is identical. When the FP is inserted (right panel), as the point source is shifted away from the focal plane, emission is rejected and is not detected by the camera, i.e. any background arriving from other planes is rejected and sectioning is achieved. The simulation yields a sectioning resolution of ˜2.2 μm, in agreement with the analytical model of equation (5) which yields ˜2.5 μm for T=0.02. The corresponding axial depth of field from equation (6) for f=250 μm,

$$\frac{Δ λ}{λ} = 0.2$$

yields 50 μm.

The following are results of the measurements and simulations performed by the inventors:

### Axial Resolution Measurements

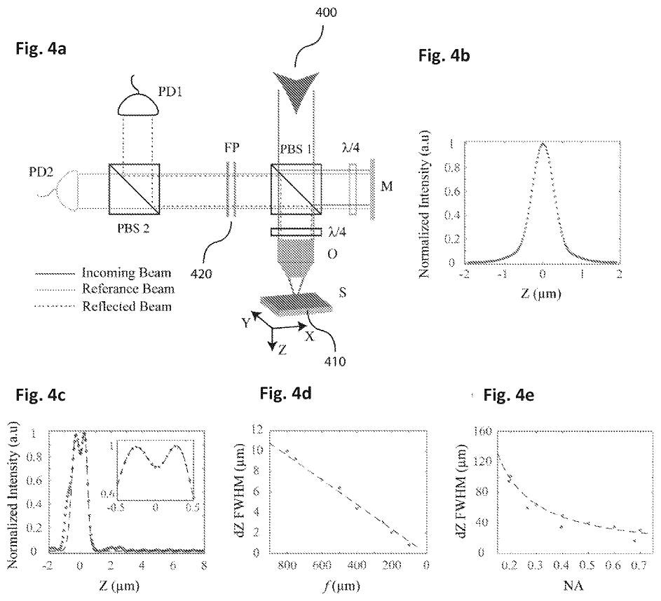

To experimentally verify their analytical model, the inventors have used the setup shown in FIG. 4a. A monochromatic collimated circularly polarized beam (red lines, 400) from a tunable laser (newport, Velocity™ TLB-6700) is split by a polarizing beam splitter (PBS 1, Thorlabs CCM1-PBS252). The transmitted beam is focused by an objective lens onto a sample placed on the three-dimensional motorized stage (410). Back reflected light (red dashed lines) is collected in an epi-detection configuration and directed through the FP (420) into a photodetector (PD1, Thorlabs DET100A). The reflected beam from PBS 1 serves as a reference arm for laser frequency locking (see methods in the Appendix), it is back reflected by a mirror (gray lines) through the FP and into another photodetector (PD2, Thorlabs DET100A). Quarter waveplates are used to modify light polarization and eliminate the need for additional beam splitters causing signal attenuation.

To measure the axial PSF, the inventors locked the laser to a resonance frequency of the FP using the signal from PD2, then the inventors placed a mirror on the translational stage and scanned it along the Z axis while recording the intensity detected by PD1 at each location along the Z axis. As discussed earlier, the axial resolution of SGM strongly depends on the lens parameters f, NA, hence, in this experiment the inventors used a self-fabricated lens (see supplementary section 2 in the Appendix) with f=100 μm and NA=0.85 as the objective lens. The FP etalon used throughout in the experiments (LightMachinery, OP-7423-6743-2) has a measured free spectral range of 15.94 GHz and Finesse of 24.77 evaluated at FWHM of the resonance peaks (see supplementary section 3 in the Appendix). FIG. 4b shows the obtained PSF with a FWHM of ˜800 nm which places the axial resolution of the system at the sub-micron regime, better than gold standard LSCM. To vividly demonstrate the ability of SGM to perform axial sectioning, a thin silicon nitride layer deposited on a silicon substrate was used. As the sample was scanned along the Z axis, two peaks corresponding to each interface of the layer were recorded (FIG. 4c, blue dots). The deposited layer thickness was 1.1 μm and the distance between the recorded peaks was measured to be 530 nm; yet, the measured result should be multiplied by the refractive index of silicon nitride ˜2 (see supplementary section 4) which gives an excellent agreement to the layer thickness.

To quantitively validate the analytical model of equation (5), in FIG. 3d the inventors compared the axial resolution dz for different focal distances at a constant NA=0.85 obtained analytically (dashed black line, T=0.02), numerically using Virtuallab Fusion (red dots) and experimentally (blue dots). Furthermore, the inventors performed a similar experiment in which the focal length of a lens was kept constant (f=4.3 mm) while the NA was varied by controlling the size of the back aperture of the objective lens (Olympus 40X, FIG. 4e). Both experiments show a good agreement between the results and the model.

### Multiplane Sectioning by a Meta-Lens

As discussed previously, the mechanism of SGM can be extended to enable multiplane single shot acquisition by introducing a meta-lens in lieu of a chromatically corrected objective. To demonstrate this concept, the inventors first designed a truncated-waveguide-based meta-lens following the hyperbolic phase function:

$$φ ( r ) = \frac{2 π}{λ} ( f - \sqrt{{r}^{2} - {f}^{2}} ) .$$

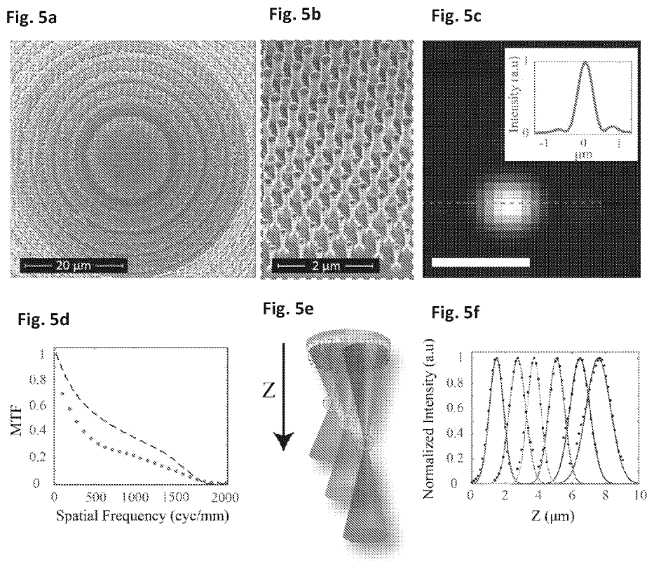

The design was carried out using a commercial software (PlanOpSim), for specific design parameters and performances see supplementary section 5 in the Appendix. The meta-lens was fabricated using a silicon-nitride on glass substrate to provide transparency in the visible regime, focal length was set to 100 μm and NA to 0.85 (for fabrication details see methods). In FIG. 5a a SEM image of a large section of the meta-lens is presented, an enlarged region marked by a red dashed line is shown in FIG. 5b.

The inventors characterized the optical performances of the lens; the PSF was measured using a high NA (0.9) objective lens to image the focal point (FIG. 5c) from which the modulation transfer function (MTF) was extracted and compared to the diffraction limited MTF (FIG. 5d, blue dots and dashed line respectively). Using the setup presented in FIG. 4a, the inventors used the meta-lens as the objective lens and applied different wavelengths (ranging from 765 nm to 790 nm) to examine the depth variations of the focal plane. As shown schematically in FIG. 4e, each wavelength is focused at a different depth, therefore, by applying each wavelength separately, SGM can provide sectioning of different planes as shown experimentally in FIG. 5f. Consequently, the entire axial information can be acquired simultaneously by introducing a broadband source, or from its own radiation if its profile is measured, as it is shown next.

### Single Shot Axial Acquisition

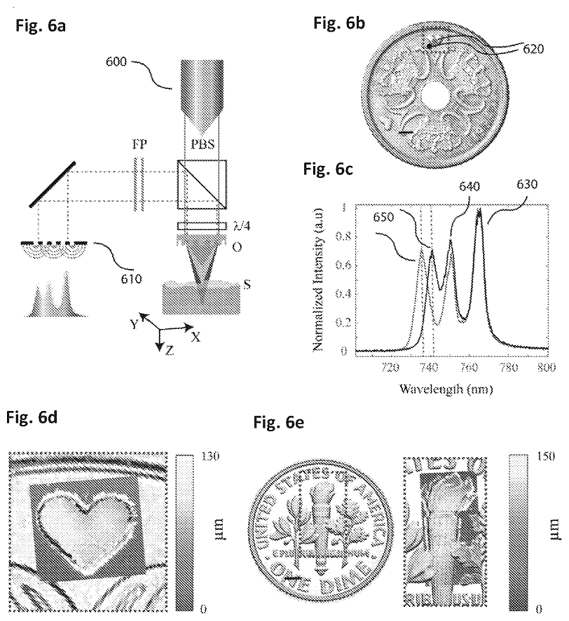

To demonstrate the capability of single shot axial imaging over a sample (rather than the Z axis scanning performed so far), the inventors modified the setup as shown in FIG. 6a. The inventors introduced a broadband light source (600) by filtering the emission spectra of a supercontinuum laser (NKT, SuperK EXTREME) to the range 700-850 nm, compatible with the FP operational range. Additionally, the inventors substituted the photodetector by a spectrometer (Ocean Optics, FLAME-T-XR1-ES, 610), thus, each spectral component acts as a photodetector for a specific axial location. For instance, a peak at a certain wavelength obtained by the spectrometer corresponds to a reflection/emission from a specific depth within the sample. The same wavelength reflected/emitted from an additional depth location is blocked by the FP in accordance with the SGM principle. Thus, there is a one-to-one correlation between the spectral measurement and the depth information which is determined by equation (6).

The inventors' first test sample of choice was a Danish krone coin shown in FIG. 6b. The coin was placed on the stage and a microscope cover slip was placed on top of it; to enable the significant depth acquisition required in this measurement, a diffractive lens was fabricated with a large focal length of 5 mm to be used as an objective lens (see supplementary section 2 in the Appendix for details). A small section of the coin was selected (FIG. 6b, dashed blue region) and the spectrums obtained from two points (620) within the region were recorded and shown in FIG. 5c. Both spectrums contain three peaks; the first (630) and second (640) peaks correspond to the reflections from the upper and lower surfaces of the cover slip respectively, hence, they appear at the same location in both spectrums. Yet, the third peak (650) attributed to the reflection from the coin, is red shifted for the black spectrum compared to the blue one due to the variation in depth of the two locations. The spectral variation can be translated into depth information either by using equation (6) or by examining the spectral distance between the first and second peaks corresponding to the known thickness of the coverslip.

Full three-dimensional scans of regions of the Danish krone and a USA dime are presented in FIGS. 6d and 6e respectively. The integration time needed to acquire the depth information at each pixel was 30 ms, however, this is not a fundamental limit by any means as the integration time depends on the available power (in the inventors' experiment ˜1 mW at the sample plane) and the sensitivity of the spectrometer. The signal-to-noise ratio measured as the standard deviation of depth variations over a flat region is ˜4 μm. Due to the reflection from the coverslip, any lateral stage tilt (which is unavoidable) is self-removed by examining the peaks separation rather than the location of a single spectral signature. It can be noted that height maps presented in FIGS. 6d and 6e cannot be recorded using an interferometric device such as an optical profilometer due to the multiple reflections from different interfaces.

### Spectrally Gated Topography (SGT)

To prove the feasibility of SGT using a simple RGB camera, the inventors simulated the setup presented in FIG. 2 using the Code V software. The inventors constructed a diffractive lens with a focal length of ˜34 cm for λ=400 nm and an aperture of 5 cm (these values can be varied to satisfy system requirements). The ray tracing result is shown in FIG. 2b, different wavelengths are focused at different planes as expected. It can be noted, that it is possible to place a stop aperture at the front focal plane of the lens to correct for coma and astigmatic aberrations, however, for this demonstration the inventors placed the aperture close to the lens since they are looking at a relatively small field of view where such aberrations are not significant. Next, the inventors placed a “white” (i.e. equal RGB values) grid object at infinity (FIG. 2c) and recorded the response of CCD1. Since the object was placed far away the camera showed a clear “blue” pattern (FIG. 2d), when the object was placed 1.35 meters away from the lens, the camera showed a “green” pattern (FIG. 2e), and as the object was brought closer to 68 cm the grid appeared “red” on the camera (FIG. 2f).

To estimate the sensitivity of the system to slight shifts in depth the inventors placed the object at a distance of 68.02 cm from the diffractive lens. Comparing the RGB values between this scenario and the 68 cm distance the inventors were able to detect changes in the RGB values of the imaged grid. Hence, even without any data processing or excessive algorithms the inventors were able to demonstrate a depth resolution of 200 μm which is comparable to state-of-the-art 3D scanners.

### Full Field SGM

To understand and quantify the capability of full field SGM, the inventors have performed a more rigorous treatment of the mathematical derivation. The normalized transmission function of the FP is given by:

$$T = \frac{1}{1 + F {sin}^{2} ( \frac{π}{λ} 2 nD cos θ )} \qquad ( 8 )$$

where

$$F = {( \frac{2 \cdot ℱ}{π} )}^{2} ,$$

[symbol]

being the finesse of the FP, n is the refractive index of the FP medium, D is the distance between the mirrors of the FP and λ is the operating wavelength. Hence, by substituting the value of θ from equation (1) into equation (8) and integrating over the entire aperture of a lens with radius R, the transmission of light through the FP can be expressed as a function of the displacement dz:

$$T ( d z ) = \frac{1}{π {R}^{2}} {\int }_{0}^{R} \frac{2 π r}{1 + {( \frac{2 \cdot ℱ}{π} )}^{2} {sin}^{2} ( \frac{π}{λ} 2 nD cos ( a tan ( \frac{r}{\frac{{f}^{2}}{dz} + f} ) ) )} d r \qquad ( 9 )$$

Equation (9) can be solved numerically, thus the axial sectioning resolution dZ can be obtained from the width of the transmission function (equation 5 is an approximation of equation 9). Since here the inventors were interested in the case of broadband illumination, the transmission ‘T’ as a function of wavelength is desirable. However, changing the wavelength also effectively changes the focal length ‘f’ which needs to be integrated into the solution. To do so, the inventors define the following:

$$λ = {λ}_{0} + Δλ \qquad ( 10 )$$

$$f = {f}_{0} \cdot ( 1 + \frac{Δ λ}{{λ}_{0}} ) \qquad ( 11 )$$

where λ₀ is the nominal wavelength (i.e. the wavelength for which the focal length is f₀) and Δλ is the change in wavelength. The inventors note that since ‘dz’ represents the shift of the source from the focal plane, it can be expressed as a function of the change in wavelength as:

$$d z = \frac{{f}_{0} \cdot Δλ}{{λ}_{0}} \qquad ( 12 )$$

Substituting equations (10), (11), (12) into (9) yields:

$$T ( Δ λ ) = \frac{1}{π {R}^{2}} {\int }_{0}^{R} \frac{2 π r}{1 + {( \frac{2 \cdot ℱ}{π} )}^{2} {sin}^{2} ( \frac{π}{{λ}_{0} + Δ λ} 2 nD cos ( a tan ( \frac{r}{\frac{{f}_{0}^{2} \cdot {( 1 + \frac{Δ λ}{{λ}_{0}} )}^{2}}{\frac{{f}_{0} \cdot Δλ}{{λ}_{0}}} + {f}_{0} \cdot ( 1 + \frac{Δ λ}{{λ}_{0}} )} ) ) )} d r \qquad ( 13 )$$

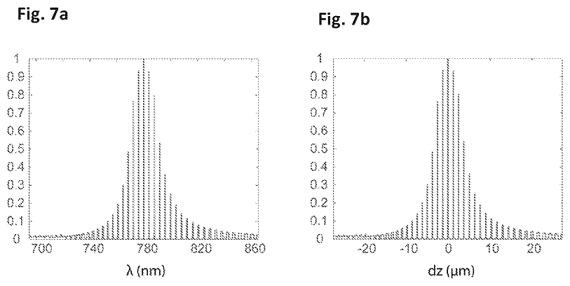

FIG. 7a shows a plot of equation (13) with the following parameters: Free-spectral-range ˜ 4 nm (corresponding to a thin 52 μm Fabry-Perot), lens NA=0.7 (corresponding to an aperture radius of 250 μm and a nominal focal length of 250 μm), Finesse=100. Clearly, wavelengths which defer from the nominal wavelength chosen to be 780 nm are greatly suppressed due to the multiangle impinging onto the Fabry-Perot surface. To emphasize the axial resolution of such system, in FIG. 7b the inventors transformed the x-axis from wavelength to axial location using equation (12). The full-width-half-maximum of the envelope function in FIG. 7b is approximately 7 μm, which means that for a multilayered scenario two layers can be clearly distinguished if they are separated by more than 7 μm. However, the accuracy of topographical measurements is orders of magnitude higher as the central location of the envelope function can be located using fitting algorithms. It is possible to increase the axial resolution by increasing the Finesse of the Fabry-Perot or the NA of the meta/diffractive lens.

### Further Details on the Aspects of the Disclosure

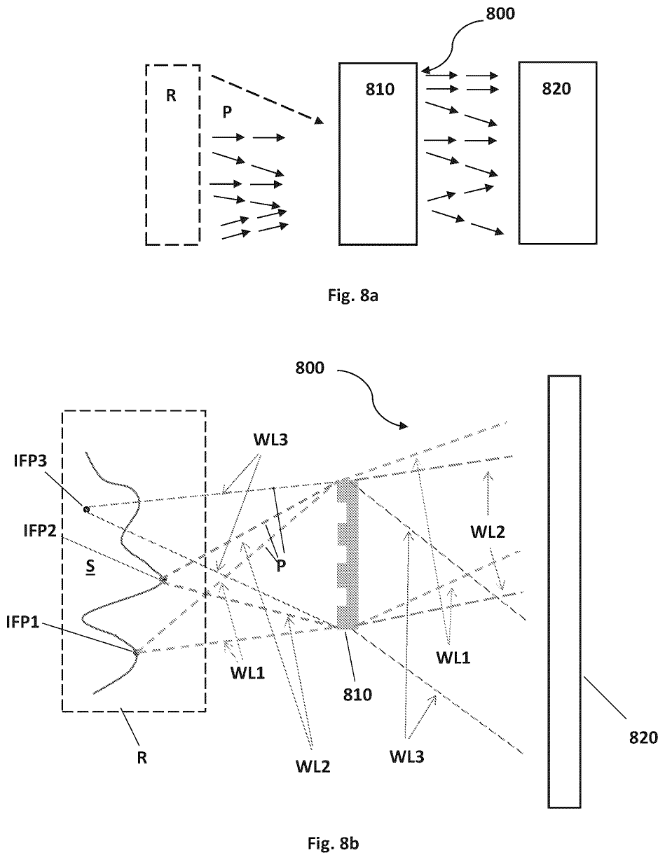

FIGS. 8a and 8b schematically illustrate a system 800 for use in optical topographical and/or tomographic 3D imaging. The system includes a lens unit 810 and an etalon structure 820. Lens unit 810 is chromatically dispersive so that its focal length varies depending on a light wavelength. It may be chromatically uncorrected or uncompensated. The lens unit 810 will pass therethrough polychromatic light P which is to arrive from and originate at a region R (where sample S is to be placed, as indicated in FIG. 8b), while selectively collimating those spectral components of the polychromatic light P which are in focus based on their wavelengths and origins in region R. For example, as schematically illustrated in FIG. 8b for a spectral component of a first wavelength WL1, a point location IFP1 on an external surface of the sample S is in focus for the lens unit 810. Similarly, for a spectral component of a second wavelength WL2, a point location IFP2 on the external surface of the sample S is in focus for the lens unit 810. For a spectral component of a third wavelength WL3, a point location IFP3 inside the sample S, which is partially transmissive above it, is in focus for the lens unit 810 (the illustration is drawn as if the refractive index inside of the sample is as outside of the sample, but this is the sake of simplicity only, without any such requirement whatsoever). The etalon structure 820 is accommodated in an optical path of light being output from the lens unit 810 to receive the collimated light. The etalon structure 820 is configured to operate with multiple resonant wavelengths and to provide spectral transmittance peaks at said resonant wavelengths (at least for some directions of the collimated light from the region R).

In some cases, the etalon structure 820 may be configured to provide a simultaneous operation of the multiple resonant wavelengths. For example, a Fabry-Perot etalon simultaneously presents a plurality of resonant wavelengths separated by a free spectral range. Additionally, the resonant wavelengths may be tunable for the collimated light which is to arrive from the lens unit 800. For example, a Fabry-Perot etalon and various others may be installed within the system 800 with at a variable angle with respect to an optical axis of the lens unit 810. Additionally, or alternatively, the etalon structure 820, be it Fabry-Perot etalon or a different etalon structure, can be for example a piezo-tunable, and/or temperature-tunable, and/or electrostatically tunable. The etalon structure 820 may have a pre-set wavelength range as one of the specifications, which by itself can determine an operating wavelength range of the system 800, or whose intersection with a nominal wavelength range of the lens unit 810 can determine the operating wavelength range of the system 800.

In some other cases, the etalon structure 820 may be tunable to operate with different resonance conditions each characterized by one of the multiple resonant wavelengths. For example, the etalon structure 820 may be presented by a guided mode resonance filter. The system 800 then may utilize wavelength sweeping for either the axial imaging with the lateral scanning or the full field (3D) imaging (microscopic or not).

The etalon structure 820 may be tunable for adapting the resonant wavelengths thereof to a range of depths of the sample.

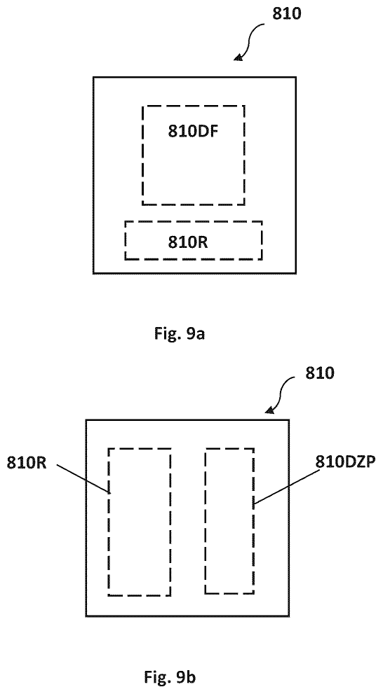

FIG. 9a schematically illustrates that the lens unit 810 may include a dispersive flat optical lens 810DF. The dispersive flat optical lens may be a diffractive lens. The diffractive lens may, instead of a convex surface of a refractive lens, have a “flattened” surface broken down into radial zones with the phase delay of modulo 2π (or a multiple thereof). In some cases, the diffractive lens may be a multi-level diffractive lens.

The dispersive flat optical lens 810DF may be a meta-lens. In a metalens, presenting a type of a metasurface, the phase may be induced via the response of nanostructures (called nanoantennas) built on the surface of the substrate material. This contrasts with a conventional diffractive lens (CDL) where the phase inducing mechanism is still like that of a refractive lens—based on the length of the ray path inside the lens material.

So far, three main methods to introduce the phase delay in dielectric metasurfaces are truncated waveguide, geometrical phase, and resonant or Huygens nanoantennas.

Additionally, or alternatively, the lens unit 810 may include a refractive lens 810R. For example, the refractive lens may be tunable. Overall, the lens unit 810 is chromatically dispersive.

FIG. 9b schematically illustrates that the lens unit 810 may include a diffractive zone plate 810DZP. Additionally, or alternatively, the lens unit 810 may include a refractive lens 810R (again, the refractive lens may be tunable), as well as the dispersive flat optical lens shown in FIG. 9a.

The phase induced by the nanoantennas can be limited in magnitude to about 2π, and a metalens of a significant optical power may be considered as a diffractive lens, as it also induces phase modulo 2π (in this specific case, not the classical definition for optical power of a lens is meant according to which it is the inverse of the focal length, but by the optical power is meant the Fresnel number of the lens, which is the maximum induced phase (of the “unwrapped” wavefront) in units of π). A metalens then often is a type of diffractive lens. However, not every diffractive lens is a metalens.

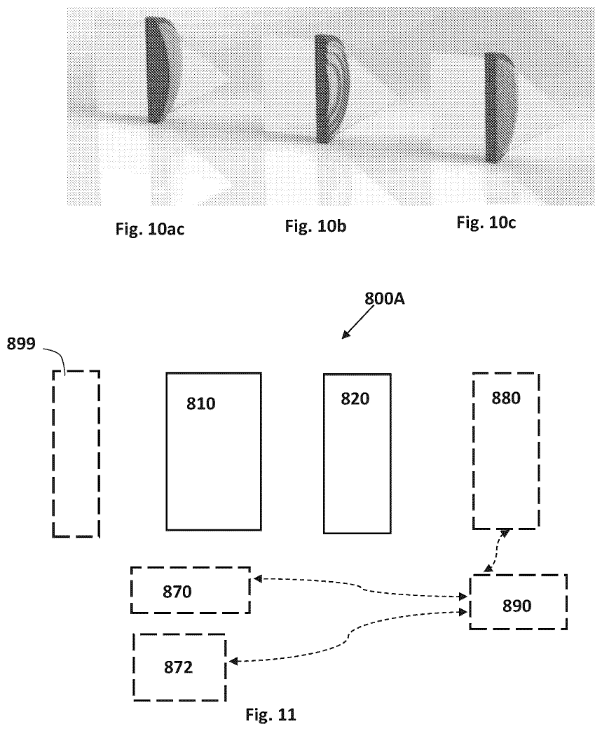

FIGS. 10(a) to 10(c) present artistic cross-sectional sketches of various lens types. FIG. 10(a) relates to a refractive lens, FIG. 10(b) relates to a diffractive lens showing flattening by division into radial zones, and FIG. 10(c) relates to a metalens showing nanoantennas for phase control.

In the system 800 the lens unit may have a focal length (at a nominal wavelength) in a range from 100 μm (microns) to 1 m, or be operable to change the focal length within this range. For some semiconductor inspection applications, the range may be more specific, for instance, from 1 mm to 5 mm: such a range may be useful for inspection of conductive vertical connections or bumps. Also, for some object scanning for 3D printers the range may be more specific, for instance, from 1 cm to 1 m.

FIG. 11 schematically illustrates an optical system 800A which is the same as the optical system 800, but further includes an optical detector 880 (optional in the system 800). This detector is configured to detect that output of the etalon structure 820 which is consequential to the polychromatic light P arriving from and originated at the sample S. In some cases, a different optical detector may be further added to the system 800 or 800A to detect the inconsequential output (for example, in the manner how the detectors PD1 and PD2 are used in FIG. 4a: PD2 is used for detecting output of the etalon structure in the reference arm inconsequential to the light emerging from the sample). Hence, the detector 880 generates measured data indicative of the detected output of the etalon structure 820.

The optical detector 880 may include a spectrometer or a spectrophotometer. The optical detector 880, or the spectrometer or spectrophotometer may include a CCD image sensor or an active-pixel sensor. Also, the optical detector implemented with the CCD or active-pixel sensor or in another way may include or be presented by a multispectral or hyperspectral camera. The multispectral camera may optionally be configured to operate with from 3 up to 30 spectral bands or other spectral modalities in each pixel. The hyperspectral camera may optionally be configured to operate with 30 to 200, or more, spectral bands or other spectral modalities in each pixel. Those spectral bands may be distributed contiguously. When the multispectral or hyperspectral camera has other spectral modalities, they may be modalities of time-domain Fourier transform imaging (such as in case of the Fourier Transform Infrared Spectroscopy, FTIR, implemented with some cameras with a useful detection range of 400 nm to 1000 nm; but the present disclosure is not limited to this spectrum, and for some applications a different spectral region may be used).

A free spectral range of the etalon structure may be larger than a spectral resolution provided by the multispectral or hyperspectral camera. For example, this can be set for the collimated light at about the nominal wavelength of the lens unit 810 arriving along the optical axis of the lens unit 810.

The lens unit 810 may provide a longitudinally chromatic aberration so that its focal length in the operating wavelength range of the system 800A would change in a spectrum detectable by the multispectral or hyperspectral camera by at least 1%, or 3%, or 10% of a nominal focal length of the lens unit 810. The operating wavelength range of the system 800A can be determined as an intersection of the operating wavelength range of the system 800 in a configuration still without the detector 880 and the operating range of the detector.

Metalenses and etalon structures can be adapted to various wavelengths. Hence, the photodetector may be configured to detect light with at least one wavelength being in a range from 300 nm to 1 mm (and even to 1 km for some applications).

The system 800A in FIG. 11 also includes such an optional component as a control unit 890. This control unit is configured and operable to receive and process the data measured by the detector 880 and calculate a distance from the lens to that location at the sample from which the detected light emerged. In particular, it may include to this end a processor (for example, a single-core processor, a multi-core processor, an application-specific integrated circuit (ASIC), a field programmable gate array (FPGA), and/or another hardware device). The calculation can be based on the spectral signal from the optical detector 880. If the optical detector has an image sensor, such as CCD or active pixel sensor or an image sensor of a different type, the calculation for a that location can be based on a spectral signal from the respective pixel of the image sensor.

Furthermore, the calculation may be based on a spectral profile of light illuminating the sample. The control unit 890 may store such information, or it may receive it from a source of illuminating light, or it may be configured even to choose the current spectral profile and send it to the source of illuminating light, which also may be included into the system 800 or 800A.

The source of illuminating light, which is an optional component of the system 800 or 800A, and is an additional or alternative to the detector 880, is schematically shown under reference numeral 870 in FIG. 11.

The control unit 890 may receive data from the detector 880 through a wired connection, or a wireless connection, or a network combining them, or in some other way (e.g. by recording the data onto a machine readable memory carrier and moving it from the detector to the control unit for reading it there).

The control unit 890 may receive and/or send data to the source 870 of illuminating light and/or a machine-readable memory 872 through a wired connection, or a wireless connection, or a network combining them, or in some other way. For example, the data transfer may be used to improve or optimize the spectral intensity distribution of the illuminating light, depending on capabilities of the source 870 for varying it.

The control unit 890 may be configured to calculate the distance based on a wavelength of an only one spectral band from spectral bands of the detector, when the spectral signal represents a detection by that only one spectral band of a part of the polychromatic light originating at the location at the sample. For example, in the very simple case if only RGB data is produced by the detector, and the spectral signal is fully R for the respective location at the sample, then the distance calculation is based on this R signal.

The calculation based on the spectral signal representing a detection by only one spectral band can be utilized extensively for the case of axial acquisition with the lateral scanning. In some embodiments, the control unit may utilize only such a calculation.

Alternatively or additionally, the control unit 890 may be configured to calculate the distance based on a wavelength of an only one spectral band from spectral bands of the detector when the spectral signal represents a detection by two spectral bands from the spectral bands of the detector of a part of the polychromatic light originating at the location at the sample, so that the calculation would be based on that spectral band which produced a relatively greater signal. Again, for example, in the very simple case if only RGB data is produced by the detector, and the spectral signal is only R and G for the respective location at the sample, then the distance calculation is based on the R signal when it is stronger and on the G signal when it is stronger (assuming the illuminating light of equal intensity in R and G and equal detection range for R and G in the pixel: clearly the selection can be adapted to choose the wavelength with the higher transmittance through the lens unit 810 and the etalon structure 820).

The calculation based on the selection of that one of two bands which has shown the relatively greater signal or corresponds to a larger transmittance through the combination of the lens unit 810 and the etalon structure 820 can be utilized, for example, in the case of the full field acquisition.

In an alternative case, the calculation of the distance may be based on a linear interpolation of the wavelength based on those two detected spectral signals, and then the determination of the distance based on the interpolated wavelength. Such a calculation can be used for example for the case of axial acquisition with the lateral scanning.

Further, alternatively or additionally, the control unit may be configured to calculate the distance by estimating a central wavelength of an envelope of a spectral distribution of a part of the polychromatic light originating at the location at the sample, when the spectral signal represents a detection by three or more of spectral bands of the detector of this part. This has been discussed for the very simple case of three bands RGB above. The peak in the envelope may be assumed to be gaussian, or it may be assumed to be of a certain different shape, depending, for example, on the dispersion profile of the lens unit.

The system 800 or 800A may further include an achromatic imaging lens unit for directing that light output of the etalon structure which is to arrive to the optical detector 880. An example of the achromatic imaging lens unit is provided by lens 340 in FIG. 3. Additionally, or alternatively, the system may include a reference arm optical detector and a reference arm achromatic imaging lens system for focusing a part of light reflected and/or emitted by the sample on the reference arm optical detector while bypassing the etalon structure. This may allow to take into account (e.g. by the normalization) that the sample demonstrates significant absorption and/or its own significant emission, if this is the case (while this absorption and/or emission may be not caused by the illuminating light from the source).

As it follows from above, the etalon structure 820 may be a Fabry-Perot etalon.

Additionally, or alternatively, the etalon structure 820 may be configured to have a finesse in a range of from 2 to 500, or from 15 to 500, or from 30 to 500, or from 15 to 250, or from 30 to 250, or from 10 to 150, or from 15 to 100, or from 25 to 75 (for example, this can be set for the normal incidence angle at about the nominal wavelength of the lens unit).

Additionally, or alternatively, the etalon structure 820 may be configured to have a free spectral range in a range of 10 nm-0.0001 nm, or 10 nm-0.001 nm, or 10 nm-0.01 nm, or 10 nm-0.1 nm (for example, this can be set for the normal incidence angle at about the nominal wavelength of the lens unit).

Additionally, or alternatively, the lens unit 810 may be configured to collect the polychromatic light from a field of view comprising angles of arrival up to 70°, or 50°, or 30°, or 10°, or 5° measured from the optical axis of the lens unit 810 (for the full-field 3D capture at one shot; for the axial capture with the lateral scanning the angles may be up to 0.1°, or possibly 1°).

As mentioned above, the system 800 or 800A may include a source of polychromatic illuminating light 870 configured to illuminate a region R where the sample S is to be placed to produce at least a part, or most of the polychromatic light P originating at the sample S (i.e. which is to re-emerge from the sample S), from an external surface of the sample and/or an internal surface of the sample and/or anything at one or more depths within the sample. This light can be the first or other order reflection from the sample. Additionally, or alternatively, the response can include scattering and/or fluorescent and/or other response, from an external surface of the sample and/or an internal surface of the sample and/or anything at one or more depths within the sample.

The source 870 of polychromatic illuminating light can include, for example, a laser, a LED, an optical fiber light source, and/or a lamp, and a light source of another type. It may be adapted to provide broadband illumination including spectral components corresponding to a plurality of the resonant wavelengths of the etalon structure (at various angles of arrival of the collimated light, whenever needed).

The broadband illumination may have more or less equal intensities at different wavelengths. Alternatively, the source 870 of polychromatic illuminating light may be adapted to provide illumination with a spectral intensity distribution peaking at a plurality of the resonant wavelengths of the etalon structure. Such an option may be especially useful for the axial acquisition with the lateral scanning.

The system 800 or 800A may include the machine-readable memory 872 storing a record on a predetermined or measured spectral profile of the polychromatic illuminating light producible by the source 870, as schematically shown in FIG. 11. The memory 872 can be an external memory with respect to the source 870 and/or the control unit 890; however, alternatively, in some embodiments it may be internal with respect to the source 870 or the control unit 890. The control unit 890, as mentioned above may access the record to obtain a spectral profile of light illuminating the sample.

The system 800 or 800A may include one or more polarizing unit, accommodated in the optical path of the illuminating light to the sample and/or in the optical path of the collimated light to the etalon structure. The examples are provided by the same polarizing beam splitter PBS1 in FIG. 4a.

The one or more polarizing unit may be configured to provide to light passing therethrough a TE-mode, or a TM mode, or a circular polarization, and for beam splitting.

Also, the system may include one or more quarter waveplates to influence the polarization. An example is provided by FIG. 4a.

The TE-mode or TM-mode may be used to influence the transmittance through the etalon structure 820. For example, it may be used to narrow the peak of angular transmittance, for example in case of the axial imaging with lateral scanning.

The system 800 or 800A may optionally include an optical splitter of any type accommodated in the optical path of light output from the etalon structure 820 and configured to split from it the consequential output of the etalon structure 820. The example is provided by the polarizing beam splitter PBS2 in FIG. 4a.

The chromatically dispersive lens unit 810 may be configured to have a longitudinally chromatic aberration satisfying at least one from an inequality

$$\vert \frac{Δ λ}{λ} \vert < 0.01 \vert \frac{Δ f}{f} \vert ,$$

an inequality

$$\vert \frac{Δ λ}{λ} \vert < 0.1 \vert \frac{Δ f}{f} \vert ,$$

an inequality

$$\vert \frac{Δ λ}{λ} \vert < 0.5 \vert \frac{Δ f}{f} \vert$$

and approximately an equality

$$\vert \frac{Δ λ}{λ} \vert = \vert \frac{Δ f}{f} \vert ,$$

for two adjacent resonant wavelengths of the etalon structure separated by a wavelength difference of Δλ, where the interval of Δλ between the two adjacent resonant wavelengths covers a nominal wavelength λ of the lens unit 810 or of the system 800 or 800A, Δλ is a difference between focal lengths of the lens unit 810 at the two adjacent resonant wavelengths, and f is a focal length of the dispersive lens unit 810 at the nominal wavelength of the system. In some embodiments, the resonant wavelengths here can be those which correspond to the normal incidence angle of the collimated light or to the incidence along the optical axis of the lens unit 810.

In some embodiments, the lens unit 810 may include an array of dispersive flat optical lenses.

The etalon structure 820 may be configured with the multiple resonant wavelengths respectively varying for a range of incidence angles of the collimated light on the etalon structure 820. For example, for the Fabry-Perot etalons, the conventional resonant wavelength are those which correspond to the normal incidence. When the incidence is not normal, the values of the resonant wavelengths shift.

Also, the etalon structure 820 may be configured with the spectral transmittance peaks respectively varying for a range of incidence angles of collimated light on the etalon structure 820.

In case of the axial distribution with lateral scanning the photodetector 880 may be a spectrometer without a plurality of pixels: this can be sufficient for detect a spectral distribution of the consequential output of the etalon structure 820.

The system 800 or 800A may further include an optional support stage 899 for supporting a sample under measurements. The system 800 or 800A may be configured and operable to affect a relative displacement in at least one lateral dimension between the stage 899 and an optical unit formed by the dispersive flat lens unit 810 and the etalon structure 820.

The system 800 or 800A (without the stage 899) in various implementations can be configured as an integrated optical unit for use in a microscope. For example, the lens unit 810 and the etalon structure 820 can be assembled in a same casing with interior isolated from the ambient light or illuminating light, with only the polychromatic light P from the sample being in the field of view of the integrated unit. Further, the integrated unit may include the optical detector 880 in the same casing. Additionally, or alternatively, it may include the achromatic imaging lens unit.

Methods, which a user of the system 800 or 800A or some other system may apply, are derivable from the above description relating to the systems, and from figures referenced therein.

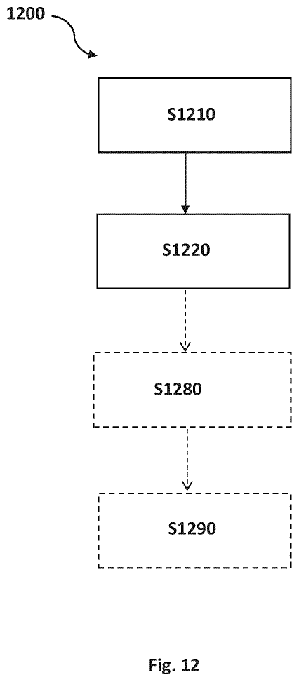

For example, FIG. 12 presents a schematic illustration of a method 1200 for use in optical topographical and/or tomographic 3D imaging of a sample. The method includes steps S1210 and S1220. Step S1210 includes passing through a lens unit, chromatically dispersive so that its focal length varies depending on a light wavelength, polychromatic light arriving from and originated at the sample, while selectively collimating those spectral components of the polychromatic light which are in focus based on their wavelengths and origins. Step S1220 includes receiving the collimated light at an etalon structure, accommodated in an optical path of light being output from the lens unit and configured to operate with multiple resonant wavelengths to provide respective spectral transmittance peaks at said resonant wavelengths to the collimated light.

Also, step S1220 may of course include passing a part of the collimated light though the etalon structure.

The method 1200 may further, optionally, include a step S1280 of detecting an output of the etalon structure consequential to the polychromatic light arriving from and originated at the sample, and generating measured data indicative of the output, with an optical detector.

Also, the method 1200 may further, optionally, include a step 1290 which including processing with a control unit the measured data to calculate a distance from the lens to a location at the sample, based on a spectral signal from the optical detector.

Additionally, or alternatively, as it follows from the above, the method in some embodiments includes illuminating, with polychromatic illuminating light, a region at the sample to produce at least a part of the polychromatic light originating at the sample as a first or other order reflection and/or scattering and/or fluorescent and/or other response from an external surface and/or an internal surface and/or one or more depths of the sample; etc.

In some implementations, the measured data may be processed for calculating the distance by a control unit, such as the control unit 890 in case of the system 800A, or a processor of the control unit, or a process of an external device.

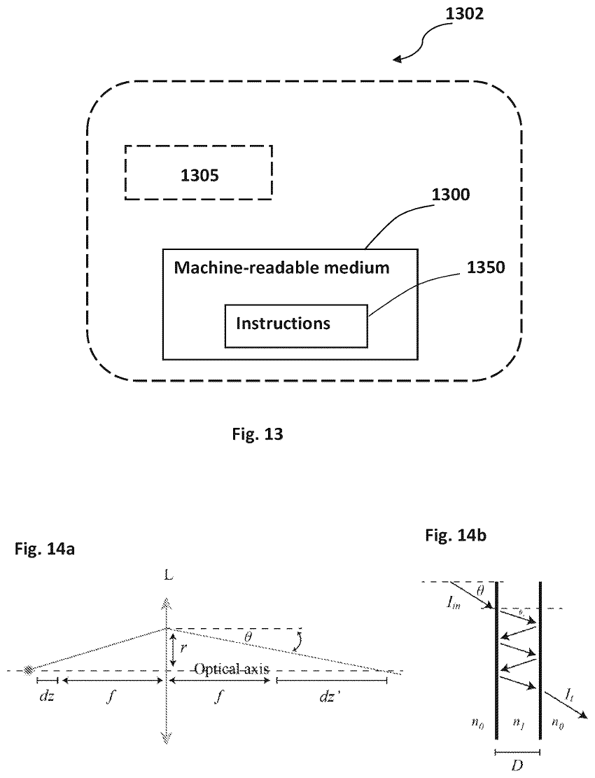

With reference to FIG. 13, there is schematically shown a non-transitory machine-readable medium 1300 (for example, the machine-readable memory carrier) storing instructions executable by a processor 1305 of a computing machine 1302. The non-transitory machine-readable medium includes instructions 1350 to calculate with the measured data, generated by any of the above methods, a distance from the lens to a location at the sample, based on a spectral signal from the optical detector as above.

In some other implementations, the above instructions may comprise further instructions, or the medium may store further instructions: to implement the respective method steps. For example, the further instructions may be adapted to make the processor to take into account a spectral profile of light illuminating the sample (which may correspond to a profile of the source 870 of the illuminating light), and/or to take into account (e.g. by the normalization) that the sample demonstrates significant absorption and/or its own significant emission, if this is the case (while this absorption and/or emission may be not caused by the illuminating light from the source; also, they may be detected through the reference arm, with the corresponding adaptation of the method).

The following includes some further details of the inventors' considerations and simulations performed by the inventors demonstrating various features of the SGM invented technique utilizing flat optics for parallel 3D imaging, in their first-person article language:

Volumetric imaging with high spatiotemporal resolution is of utmost importance for various applications ranging from aerospace and defense to real time imaging of dynamic biological processes. To facilitate three-dimensional sectioning, current conventional technology relies on mechanisms to reject light from adjacent out-of-focus planes either spatially or by other means. Yet, the combination of rapid acquisition time and high axial resolution is still elusive, motivating a persistent pursuit of novel imaging approaches. Here, the inventors introduce a new concept named spectrally gated microscopy (SGM) which enables a single shot interrogation over the full axial dimension while maintaining a sub-micron sectioning resolution. SGM utilizes two features enabled by flat optics (i.e. meta and diffractive lenses) namely—a short focal length and strong chromatic aberrations. Using SGM the inventors demonstrate three-dimensional imaging of millimeter-scale samples while scanning only the lateral dimension, presenting a significant advantage over state-of-the-art technology.

Optical microscopy has experienced a renaissance in the past decade greatly stimulated by the introduction of super-resolution modalities. Localization microscopy and structural illumination microscopy are nowadays widely available providing nanoscale lateral resolution, while other techniques based on innovative material structuring are constantly being developed. However, in many cases the depth information is of great interest and requires an additional scan over the axial dimension. Such process results in an unfortunate compromise between spatial and temporal resolution; indeed, when a large volume is of interest one needs to either sample it with high spatial resolution at the expenses the of temporal resolution or vice versa. Such a tradeoff is often intolerable and renders the modality unsuitable for many applications such as LiDAR or developmental biology where dynamic three-dimensional scenes are of interest.

Thus, three-dimensional sectioning at high spatiotemporal resolution remains the Achilles heel of optical imaging due to the lack of combined axial sub-micron resolution together with a rapid acquisition time. In this context, laser scanning confocal microscopy (LSCM) and multiphoton microscopy (MPM) have been the primary tools for decades to enable three-dimensional imaging with axial resolution up to ˜2 μm. Nevertheless, the time-consuming physical scan over all three dimensions as well as the high energy required to operate MPM has rendered these tools unsuitable for real-time volumetric imaging often required for biological dynamic studies. Recently there have been several intriguing attempts to overcome the aforementioned barrier by using confocal configurations and introducing a series of reflecting pinholes conjugated to different sample plans, or by using chromatic dispersion to obtain axial sectioning. Yet, due to the inherent confocal mechanism, a careful adjustment of the pinholes is required to meet the conjugation demand which limits the number of planes imaged simultaneously and necessitates a cumbersome alignment procedure.

By selectively illuminating a single plane at a time, light sheet microscopy and the more recent HiLO microscopy present a significant speed advantage. However, they require high optical clarity of the sample and typically achieve a lower axial resolution. Optical coherence tomography (OCT) utilizes low coherence sources to enable axial sectioning and is the gold-standard tool for retinal imaging, yet, both lateral and axial resolution are compromised to support a significant depth range thus rarely exceeding ˜5 μm. The quest for volumetric microscopic imaging techniques stimulated a variety of new ideas; light-field advanced modalities, reverberation microscopy or diffuser assisted computational reconstruction where recently demonstrated. While all these emerging strategies are intriguing, they often come with a fundamental trade-off between axial and lateral resolution or require some a-priori knowledge of the sample. Thus, instantaneous depth information is yet a sought-after goal pursued by many to facilitate real-time volumetric imaging.

Here the inventors present a new paradigm for three-dimensional sectioning named spectrally gated microscopy (SGM); as opposed to spatial (LSCM, MPM) or coherent\time (OCT) sectioning mechanisms, SGM rejects out-of-focus light through a resonance mismatch, i.e. spectral rejection. The inventors have shown that the spectral gating mechanism can provide sub-micron axial sectioning, with resolution higher than offered by state-of-the-art technology, together with a single-shot axial acquisition which eliminates the need for depth scanning. The great advantage of this modality arises from the transformation of information from the spatial domain to the spectral one, in which chromatic multiplexing can be realized by exploiting the chromatic aberrations of flat optical components such as meta-lenses. The combination of the SGM mechanism with meta-lens technology enables parallel multiplane imaging and overcomes the spatiotemporal barrier imposed by the requirement for imaging large volumes at high resolution.

The principle of SGM is presented schematically in FIG. 1a. The system encompasses a monochromatic light source, an imaging system and a high-finesse Fabry-Perot (FP) etalon. Light emitted by the source when located at the focal spot of the lens will exit the back aperture as a collimated beam. Hence, by tuning the FP resonance it is straightforward to ensure the full transmission of said emission. However, any additional light emerging from a different plane rather than the focal plane will exit the back aperture of the lens as a converging or diverging beam. Therefore, it will not comply with the pre-tuned resonance and will be rejected by the etalon. The SGM mechanism ensures that only light originating from the focal plane of the system arrives at the detector/camera. This is accomplished via angular rejection rather than the spatial one offered by confocal configurations. The SGM mechanism can be further extended to enable single shot acquisition of the entire axial axis by introducing polychromatic light and a flat optical component (i.e. a metalens) to support many focal planes.

The principle of single shot SGM using a metalens is presented in FIG. 1b. The multifocal planes provided by the meta-lens enable the spectral decoding of information from different planes. Hence, by separating and analyzing each wavelength individually using a spectrometer, sectioning of different planes is achieved simultaneously without the need for depth scanning with an adjustable confocal aperture. The inventors note that the lateral dimension still needs to be scanned, similar to other modalities.

With regard to FIG. 1, which was also mentioned in the preceding sections of the description, it should be mentioned that it equally relates to (a) Schematic illustration of the SGM mechanism with monochromatic light. A point source is imaged by a pair of lenses (L) onto a camera (C) through a Fabry-Perot etalon (FP). (b) Schematic illustration of the SGM mechanism with several point sources located at various axial locations. Light from a point source will be transmitted through the FP only if the distance from the meta-lens coincides with the focal length at the given wavelength of the source. The reflected light is analyzed after being dispersed by a spectrometer (S). (c) Optical diagram showing the ray propagation from an out-of-focus location. (d) A simulated three-dimensional PSF of the system presented in (a) without (left) and with (right) the FP etalon, insets show a YZ cross-section of the PSF (scale-bar=1 μm).

To evaluate the axial sectioning resolution provided by SGM, the inventors considered a point source shifted by dz along the optical axis and away from the focal point (FIG. 1c, for a full derivation see supplementary section 1). The rays emerging from the point source will exit the back aperture of the lens with an angle:

$$θ = a tan ( \frac{r}{\frac{{f}^{2}}{dz} + f} ) \qquad ( 1 )$$

where r is the distance from the center of the lens to the intersection of the ray with the lens surface, and f is the lens focal length. The characteristics of the FP will determine whether light arriving with angle θ will be rejected; the angle θ corresponding to a transmission value T through the FP is given by:

$$θ = a cos [ \frac{λ}{2 π nD} \cdot [ {( - 1 )}^{m} \cdot a sin ( \sqrt{\frac{1 - T}{4 T / {π}^{2} \cdot {F}^{2}}} ) + m π ] ] \qquad ( 2 )$$

where λ is the incident wavelength, D, n, F are the FP thickness refractive index and finesse respectively. Comparing the right-hand side of equations (1), (2) the axial resolution dz can be expressed as:

$$d z = \frac{{Kf}^{2}}{r - fK} \qquad ( 3 )$$

where K is a constant determined by the FP characteristics:

$$K = tan ( a cos [ \frac{λ}{2 π nD} \cdot [ {( - 1 )}^{m} \cdot a sin ( \sqrt{\frac{1 - T}{4 T / {π}^{2} \cdot {F}^{2}}} ) + m π ] ] ) \qquad ( 4 )$$

In this derivation there are two free parameters, T and r which together determine the contrast demand imposed upon the system. The value of T defines the threshold under which transmission is considered negligible, while r determines the radius at the lens plane from which light is blocked (i.e 0<r<Rₗₑₙₛ). For instance, by setting r=0.32·R where R is the radius of the lens, all photons impinging on the lens aperture beyond the radial location of 0.32·R will be blocked, corresponding to ninety percent of the lens area. Substituting the said value into equation (3) and introducing the numerical aperture (NA) in lieu of R yields:

$$d z = \frac{Kf \cdot \sqrt{1 - N {A}^{2}}}{0 \text{.32} \cdot NA - K \cdot \sqrt{1 - N {A}^{2}}} \qquad ( 5 )$$

In order for SGM to enable a multiplane single shot acquisition, the naturally large chromatic aberrations provided by meta-lenses are required. It is well known that the change in focal length of a diffractive lens and particularly metalenses is related to the wavelength shift by:

$$\frac{Δ λ}{λ} = \frac{Δ f}{f} \qquad ( 6 )$$

where λ is the designed nominal wavelength. From equation (6) the axial field of view can be expressed as:

$$Δ Z = f \frac{Δ λ}{λ} ,$$

hence, for a broadband source, a significant axial range can be covered by focal points of different wavelengths, which can then be spectrally dispersed and analyzed separately. Assuming a high finesse FP, the value of K is very small and equation (3) can be approximated as:

$$d z = \frac{{Kf}^{2}}{r}$$

and dividing the two expressions yields:

$$\frac{Δ Z}{dz} = \frac{R \cdot Δ λ}{f \cdot K λ} \approx NA \cdot \frac{Δ λ}{K λ} \qquad ( 7 )$$

Equation (7) indicates that the number of sectioned planes contained within the axial field of view for given values of K, λ, Δλ is determined by the NA of the metalens: Higher NA yields more sectioned planes within the axial field of view. It should be noted that this derivation is geometric in nature. Diffraction effects become significant particularly at high NA values and should be accounted for (see supplementary section 1).

To demonstrate the powerful effect of SGM, the inventors performed a simulation using the Virtuallab Fusion software which offers a full field-propagating tool (FIG. 1c, see methods). The exact scenario of FIG. 1a was simulated, i.e. a monochromatic emitting point source was shifted along the optical axis while the intensity distribution at every location was imaged and recorded by the camera. The specific parameters of the simulation were: f=250 μm, D=6.743 mm, F˜30, NA=0.7. As can be seen in the left panel of FIG. 1d, in the absence of the FP a typical point-spread-function (PSF) is obtained, light is not rejected and therefore the total energy at each plane is identical. When the FP is inserted (right panel), as the point source is shifted away from the focal plane, emission is rejected and is not detected by the camera, i.e. any background arriving from other planes is rejected and sectioning is achieved. The simulation yields a sectioning resolution of ˜2.2 μm, in agreement with the analytical model of equation (5) which yields ˜2.5 μm for T=0.02. The corresponding axial depth of field from equation (6) for f=250 μm,

$$\frac{Δ λ}{λ} = 0.2$$

yields 50 μm.

### Results

### Axial Resolution Measurements

To experimentally verify the analytical model used by the inventors, the setup shown in FIG. 4a was used. A monochromatic collimated circularly polarized beam (red lines) from a tunable laser (newport, Velocity™ TLB-6700) is split by a polarizing beam splitter (PBS 1, Thorlabs CCM1-PBS252). The transmitted beam is focused by an objective lens onto a sample placed on the three-dimensional motorized stage. Back reflected light (red dashed lines) is collected in an epi-detection configuration and directed through the FP into a photodetector (PD1, Thorlabs DET100A). The reflected beam from PBS 1 serves as a reference arm for laser frequency locking (see methods), it is back reflected by a mirror (gray lines) through the FP and into another photodetector (PD2, Thorlabs DET100A). Quarter waveplates are used to modify light polarization and eliminate the need for additional beam splitters causing signal attenuation.

To measure the axial PSF, the laser was locked to a resonance frequency of the FP using the signal from PD2, and then a mirror was placed on the translational stage and scanned it along the Z axis while recording the intensity detected by PD1 at each location along the Z axis. As discussed earlier, the axial resolution of SGM strongly depends on the lens parameters f, NA, hence, in this experiment a self-fabricated lens was used (see supplementary section 2) with f=100 μm and NA=0.85 as the objective lens. The FP etalon used throughout this paper (LightMachinery, OP-7423-6743-2) has a measured free spectral range of 15.94 GHz and Finesse of 24.77 evaluated at FWHM of the resonance peaks (see supplementary section 3). FIG. 4b shows the obtained PSF with a FWHM of ˜800 nm which places the axial resolution of the system at the sub-micron regime, better than gold standard LSCM. To vividly demonstrate the ability of SGM to perform axial sectioning, a thin silicon nitride layer deposited on a silicon substrate was used. As the sample was scanned along the Z axis, two peaks corresponding to each interface of the layer where recorded (FIG. 4c, blue dots). The deposited layer thickness was 1.1 μm and the distance between the recorded peaks was measured to be 530 nm; yet, the measured result should be multiplied by the refractive index of silicon nitride ˜2 (see supplementary section 4) which gives an excellent agreement to the layer thickness.

With regard to FIG. 4, which was also mentioned in the preceding sections of the description, it should be mentioned that it equally relates to (a) Optical setup: Light is focused by an objective lens (O) onto the sample (S) and collected in a reflection mode through the FP into a photodetector (PD1). Another path is used as a reference arm directed into a second photodetector (PD2) to lock the laser frequency and avoid drifts throughout the experiment. Polarizing beam splitters (PBS) and quarter waveplates (λ/4) are used to separate the paths. (b) An axial PSF showing the sectioning capability of the system. (c) An axial scan of a sample containing a thin silicon nitride layer deposited on top of a silicon substrate. (d) Axial resolution for a constant NA (=0.85) at various focal lengths, experimental results (blue dots), simulated values (red dots) and analytical prediction (black dashed line) are in good agreement. (e) Axial resolution for a constant focal length (f=4.3 mm) at various NA values, experimental results (blue dots), simulated values (red dots) and analytical prediction (black dashed line) are in good agreement.

To quantitively validate the analytical model of equation (5), in FIG. 4d the axial resolution dz is compared for different focal distances at a constant NA=0.85 obtained analytically (dashed black line, T=0.02), numerically using Virtuallab Fusion (red dots) and experimentally (blue dots). Furthermore, a similar experiment was performed in which the focal length of a lens was kept constant (f=4.3 mm) while the NA was varied by controlling the size of the back aperture of the objective lens (Olympus 40X, FIG. 4e). Both experiments show a good agreement between the results and the model.

### Multiplane Sectioning by a Meta-Lens

As discussed previously, the mechanism of SGM can be extended to enable multiplane single shot acquisition by introducing a meta-lens in lieu of a chromatically corrected objective. To demonstrate this concept, the inventors first designed a truncated-waveguide-based meta-lens following the hyperbolic phase function:

$$φ ( r ) = \frac{2 π}{λ} ( f - \sqrt{{r}^{2} - {f}^{2}} ) .$$

The design was carried out using a commercial software (PlanOpSim), for specific design parameters and performances see supplementary section 5. The meta-lens was fabricated using a silicon-nitride on glass substrate to provide transparency in the visible regime, focal length was set to 100 μm and NA to 0.85 (for fabrication details see methods). In FIG. 5a a SEM image of a large section of the meta-lens is presented, an enlarged region marked by a red dashed line is shown in FIG. 5b.

With regard to FIG. 5, which was also mentioned in the preceding sections of the description, it should be mentioned that it equally relates to: (a) A partial SEM image of the meta lens. (b) An enlarged region marked by a red dashed line in (a). (c) The PSF of the meta-lens, scale bar=500 nm. The inset shows a cross-section of the PSF. (d) The experimental MTS (blue dots) compared to the diffraction limited MTF (black dashed line) obtained by Virtuallab Fusion. (e) Schematics of the spectral focusing dispersion generated by the meta-lens, lower wavelengths are focused closer to the lens surface. (f) Axial sectioning of different planes is obtained by wavelength variation. The left (i.e. smallest axial values) peak correspond to λ=790 nm and each peak thereafter was obtained by decreasing the wavelength by 5 nm. Experimental smoothed data are shown in black dots and gaussian fits in colored lines.

The inventors characterized the optical performances of the lens; the PSF was measured using a high NA (0.9) objective lens to image the focal point (FIG. 5c) from which the modulation transfer function (MTF) was extracted and compared to the diffraction limited MTF (FIG. 5d, blue dots and dashed line respectively). Using the setup presented in FIG. 4a, the meta-lens was used as the objective lens and applied different wavelengths (ranging from 765 nm to 790 nm) to examine the depth variations of the focal plane. As shown schematically in FIG. 5e, each wavelength is focused at a different depth, therefore, by applying each wavelength separately, SGM can provide sectioning of different planes as shown experimentally in FIG. 5f. Consequently, the entire axial information can be acquired simultaneously by introducing a broadband source as is shown next.

### Single Shot Axial Acquisition

To demonstrate the capability of single shot axial imaging over a sample (rather than the Z axis scanning performed so far), the setup was modified as shown in FIG. 6a. A broadband light source was introduced by filtering the emission spectra of a supercontinuum laser (NKT, SuperK EXTREME) to the range 700-850 nm, compatible with the FP operational range. Additionally, the photodetector was substituted by a spectrometer (Ocean Optics, FLAME-T-XR1-ES), thus, each spectral component acts as a photodetector for a specific axial location. For instance, a peak at a certain wavelength obtained by the spectrometer corresponds to a reflection/emission from a specific depth within the sample. The same wavelength reflected/emitted from an additional depth location is blocked by the FP in accordance with the SGM principle. Thus, there is a one-to-one correlation between the spectral measurement and the depth information which is determined by equation (6).

The first test sample of choice was a Danish krone coin shown in FIG. 6b. The coin was placed on the stage and a microscope cover slip was placed on top of it; to enable the significant depth acquisition required in this measurement, a diffractive lens was fabricated with a large focal length of 5 mm to be used as an objective lens (see supplementary section 2 for details). A small section of the coin was selected (FIG. 6b, dashed blue region) and the spectrums obtained from two points within the region were recorded and shown in FIG. 6c. Both spectrums contain three peaks; the first and second peaks correspond to the reflections from the upper and lower surfaces of the cover slip respectively, hence, they appear at the same location in both spectrums. Yet, the third peak attributed to the reflection from the coin, is red shifted for the black spectrum compared to the blue one due to the variation in depth of the two locations. The spectral variation can be translated into depth information either by using equation (6) or by examining the spectral distance between the first and second peaks corresponding to the known thickness of the coverslip.

With regard to FIG. 6, which was also mentioned in the preceding sections of the description, it should be mentioned that it equally relates to (a) Single shot axial acquisition setup. A broadband source is focused by a flat lens onto the sample; the reflected light is collected and directed through the FP into a spectrometer. (b) A Danish krone (scale bar=1 mm). A region containing a heart shape is selected (blue dashed mark) and two points of interest having different surface elevation are selected (blue and black dots). (c) The spectrums obtained from the selected locations in (b). (d) A tomographic image of the heart region. (e) A USA dime coin (scale bar=1 mm) and the corresponding tomographic image of a selected region (blue dashed mark).

Full three-dimensional scans of regions of the Danish krone and a USA dime are presented in FIGS. 6d and 6e respectively. The integration time needed to acquire the depth information at each pixel was 30 ms, however, this is not a fundamental limit by any means as the integration time depends on the available power (in the experiment ˜1 mW at the sample plane) and the sensitivity of the spectrometer. The signal-to-noise ratio measured as the standard deviation of depth variations over a flat region is ˜4 μm. Due to the reflection from the coverslip, any lateral stage tilt (which is unavoidable) is self-removed by examining the peaks separation rather than the location of a single spectral signature. It is noted that height maps presented in FIGS. 6d and 6e cannot be recorded using an interferometric device such as an optical profilometer due to the multiple reflections from different interfaces.

Thus, the inventors have demonstrated a new concept for three-dimensional sectioning based on spectral filtering rather than spatial filtering (LSCM, Light-sheet microscopy), coherence gating (OCT) or photon statistics (MPM). The SGM mechanism when combined with the superior performances of novel flat optical components allows information from a large axial range to be simultaneously recorded, thus, the number of dimensions to be scanned is reduced to merely the lateral one. The inventors attained an axial scanning resolution up to ˜800 nm which can be further improved by introducing a higher finesse FP or reducing the focal length of the objective lens—a feature which is enabled by the introduction of flat components. To further enhance the volumetric acquisition time, it is possible to introduce a meta-lens microarray to probe the sample at many lateral locations. Such configuration will require a spectrometer to measure the output of each lens within the microarray and can be realized using miniaturized spectrometers. The trade-off between axial resolution and depth of field presented in equation (7) can be relaxed by meta-lens dispersion engineering to enable even larger chromatic aberrations.

In the above-described experiments, a label-free reflection mode was used in which the image contrast is a product of refractive index variations, however, there is no fundamental obstacle preventing the method from being applied to optical fluoresce microscopy. In all scenarios one needs to consider the total throughput of the system; in the reflection mode the main losses are attributed to the high finesse FP which provides ˜30% transmission at resonance and the meta-lens which has an efficiency of ˜20%. When labeled samples are of interest, an additional loss is expected due to the mismatch between the broad emitted spectrum and the plane of interest (only particular wavelengths are transmitted from each plane).

In order to obtain three-dimensional images, in this example the sample was physically scanned using a translational stage (Prior H107_NB) over the region of interest. Instead, scanning the beam over the lateral dimension is preferable from an acquisition speed perspective, however, such operation mode is infeasible due to the limited field-of-view provided by high NA meta/diffractive lenses. With the great advances in flat optical technologies many current studies are recently dedicated to the performance enhancement of flat optical components which can aid the emergence of SGM as a powerful rapid three-dimensional optical tool.

### Methods

Optical simulations: To simulate the optical performances of SGM the field propagation module of Virtuallab Fusion 2020.2 was used. A point source with a diameter of 1 nm was generated at the focal plane of an ideal lens with a given aperture. A second ideal lens was placed at an arbitrary distance away from the first one, and a camera detector was placed at the focal plane of the second lens. Due to the identical focal lengths of the lenses, the system images the point source onto the camera with a magnification of 1. In between the two lenses, a FP composed of two identical stratified media surfaces was placed, the transmission spectrum was set using a built-in highly reflective coating. First, a parameter run was performed on the source wavelength to find a resonance peak with high accuracy (˜0.1 μm), from this scan the finesse and FSR of the system were also evaluated. Next, the source wavelength was set to be at the said resonance and performed another parameter run this time over the location of the first lens. By recording the intensity distribution at the camera detector for each location of the lens the results shown in FIG. 1(d) and FIGS. 4(d-e) were obtained.

Laser locking: To lock the laser frequency and avoid drifting throughout the measurement a standard wavelength modulation protocol was used. In short, the laser frequency was modulated with an amplitude of ˜10 MHz about the central output wavelength at a frequency of 1 kHz. The frequency modulation is achieved by applying a sinusoidal voltage signal to the laser piezo which is supplied by a lock-in amplifier (Zurich Instruments, UHFLI). Next, the central output frequency was tuned using an external function generator to match the frequency of a resonance peak of the FP. This was done by recording the intensity from PD2; As the wavelength is tuned across the FP resonance the frequency modulation results in a modulated signal output from PD2 with a varying phase. When one slope of the resonance was tuned to, a signal with a negative phase was obtained, and when the other slope was tuned to a signal with a positive phase was obtained, and when at the resonance a signal with a frequency of 2 kHz and a phase of zero was obtained. This signal was fed to the lock-in amplifier, which operates as a phase detector, and receive an error signal. The error signal is fed to an integrator (Zurich Instruments, UHFLI), the integrated error is fed back to the laser ensuring the error will be zero and the laser will remain on resonance

Meta-lens fabrication: To fabricate the meta-lens, first a 1.1 μm thick silicon-nitride was deposited on a glass substrate using PECVD (Oxford Instruments). Next, the substrate was coated with two layers of PMMA—the first layer was PMMA 450 with a thickness of 300 nm and the second layer was PMMA 950 with a thickness of 100 nm. E-beam lithography (Elionix) was used to transfer the lens pattern to the PMMA resist; the exposed regions were removed after development. To etch the Si₃N₄, an alumina mask was used being defined by evaporating a 50 nm layer of alumina followed by the removal of the PMMA (liftoff process). Finally, the sample was etched by RIE (Corial 210-RL) leaving only the pillar region which was protected by the alumina mask.

### Supplementary Material

### 1. Mathematical Derivation

To derive the equations presented above, the optical diagram shown in FIG. 14(a) was considered. If a point source is located on the optical axis and shifted by dz away from the focal plane of a lens (L), any ray emerging from the point source will refract/diffract and exit the back aperture of the lens with an angle θ depending on the location of the impinging ray (r). The point source will be imaged by the lens at a location dz′ away from the back focal plane, the relation between the object and imaged planes can be described by the Newtonian form of the imaging equation:

$$dz \cdot {dz}^{′} = {f}^{2} \qquad ( S1 )$$

From a simple geometrical consideration, the angle θ can be related to the length scales of the problem:

$$tan ( θ ) = \frac{r}{f + {dz}^{′}} \qquad ( S2 )$$

Substituting dz′ from equation (S1) into (S2), one arrives at equation (1) presented above:

$$θ = a tan ( \frac{r}{\frac{{f}^{2}}{dz} + f} ) \qquad ( S3 )$$

The geometrical postulation ignores diffraction effects; to account for such effects one can assume an uncertainty regarding the source location (arising from the diffraction limit of the imaging lens) which results in an uncertainty of the refraction angle θ. Given a diffraction limited lens, the point source at the image plane is magnified by f/dz and results in a spot of size of

$$∼ \frac{λ r}{f} \cdot \frac{f}{dz}$$

at the object plane. Therefore, the value of r in equation (S2) should be replaced by

$$r \pm \frac{λ r}{dz}$$

and equation (S3) becomes:

$$θ = atan ( \frac{r \pm \frac{λ \cdot r}{dz}}{\frac{{f}^{2}}{dz} + f} ) \qquad ( S4 )$$

Note, that for values of dz which are much larger than λ, the difference between equations (S3) and (S4) is negligible.

FIG. 14b shows: (a) A schematic ray propagation diagram. (b) A schematic transmission diagram through a FP.

As the ray enters the FP, it is refracted according to Snell's law and propagates within the device at an angle:

$${θ}_{t} = asin ( \frac{{n}_{0}}{{n}_{1}} sin θ ) .$$

The transmitted light can be calculated by summing the series of scalar amplitudes of the reflected and transmitted waves from the two interfaces of the FP. By denoting the accumulated phase arising from an optical path difference between adjacent rays as:

$$δ = \frac{2 π}{λ} 2 {n}_{1} D cos ( {θ}_{t} )$$

the transmittance spectra through the FP can be expressed as:

$$T = {I}_{t} = \frac{1}{1 + F {sin}^{2} ( \frac{δ}{2} )} \qquad ( S5 )$$

where

$$F = {( \frac{2 \cdot ℱ}{π} )}^{2} ,$$

[symbol]

being the finesse of the FP, and the total transmittance is normalized to the value of 1. From equation (S5) and the expression for δ it is straightforward to evaluate the propagation angle θₜ as a function of the transmittance T:

$${θ}_{t} = acos [ \frac{λ}{2 π n D} \cdot [ {( - 1 )}^{m} \cdot asin ( \sqrt{\frac{1 - T}{\frac{4 T}{{π}^{2}} \cdot {ℱ}^{2}}} ) + m π ] ] \qquad ( S6 )$$

For small values of θ, from equation (S4) the value of θₜ can be approximated based on Snell's law as:

$${θ}_{t} = \frac{{n}_{0}}{{n}_{1}} atan ( \frac{r \pm \frac{λ \cdot r}{dz}}{\frac{{f}^{2}}{dz} + f} ) \qquad ( S7 )$$

Comparing the right-hand sides of equations (S6), (S7) one arrives at the analytical expression for the axial resolution:

$$dz = \frac{K {f}^{2} + λ \cdot r}{r - fK} \qquad ( S8 )$$

From a physical point of view this means that the value of dz is bounded by the wavelength, i.e. even for very small values of f, the axial resolution of SGM cannot surpass the wavelength.

### 2. Diffractive Lens Design

To design the diffractive lenses used throughout this work, the inventors first used CODE V to obtain the coefficients of a phase polynomial which satisfies the lens requirements. The number of coefficients was increased until the MTF of the designed component coincided with the diffraction limited MTF. Next, a modulo 2π operation was performed on the obtained polynomial, to give a quasi-periodic saw-tooth structure. This structure was then binarized to give phase values of 0, π solely. Accordingly, the material thickness was selected to provide a phase delay of π at unperturbed locations and 0 when fully etched. Here, the inventors decided to use a thin layer of amorphous silicon on a glass substrate; silicon has a high refractive index, hence a thin layer of 140 nm is sufficient to attain a π phase delay. For such a thin layer, absorption at λ>700 nm is negligible.

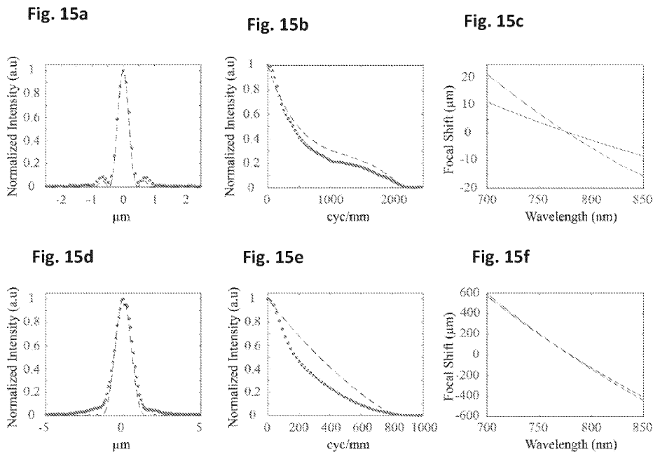

Here, the inventors present the characteristics of two exemplary lenses used in this work. For the lens used in FIG. 4b (NA=0.85, f=100 μm), FIGS. 15(a-c) show the PSF, MTF and chromatic focal length shift respectively. Experimental data are presented as blue dots and the Code V simulated results are denoted with a black dashed line. The chromatic focal shift is presented either by considering the paraxial focus (blue line) or the wave front optimized best focus location (red line). FIGS. 15(d-f) shows equivalent results for the lens used in FIG. 6 (NA=0.35, f=5 mm).

FIGS. 15a-f show: (a-c) PSF, MTF and chromatic focal shift of the diffractive lens used in FIG. 2b. (d-f) PSF, MTF and chromatic focal shift of the diffractive lens used in FIG. 4. Measured data (blue dots) and simulated results (black dashed line) are in good agreement. Chromatic focal shift is calculated by the paraxial approximation (blue line in c,f) or by wave front best focus evaluation (red line in c,f).

### 3. Fabry-Perot Characterization

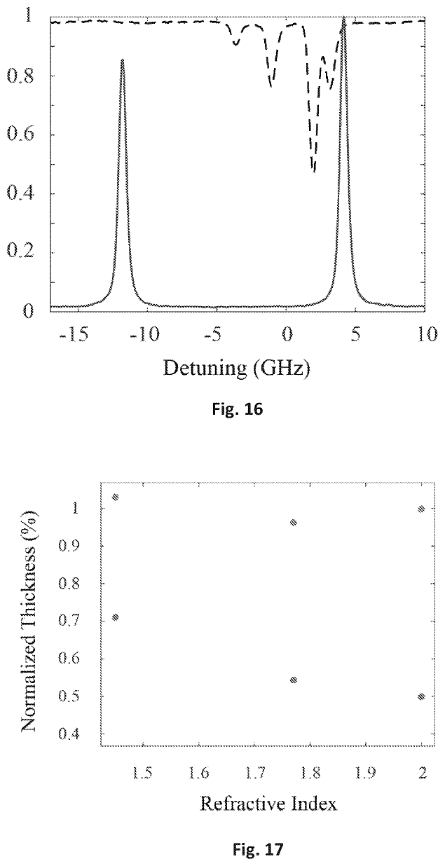

FIG. 16 shows: Transmission spectra through the Rubidium vapor cell (black dashed line) and through the FP (blue line)

To measure the FSR and finesse of the FP, the laser beam was frequency modulated around λ=780 nm and split into two arms. The first arm was transmitted through a Rubidium vapor cell (Thorlabs, GC19075-RB); the recorded spectrum showing the absorption signature of the Rubidium D₂ line is shown in FIG. 16, black dashed line. The second arm was transmitted through the FP, the output is shown as the blue solid line in FIG. 16. From the known spectral gap between the D₂ Rubidium 85 absorption peaks (˜3.035 GHz), the inventors calibrated the frequency axis and evaluated the FSR as well as the FP peak width from which the finesse was extracted. The measured values were: FSR=15.94 GHz, Finesse=24.77.

### 4. Depth Dependence on the Refractive Index

FIG. 17 shows: The relative measured thickness (blue dots) and the corrected measured thickness obtained after multiplication by the refractive index (red dot).

The depth measured by all optical modalities is affected by the refractive index of the interrogated layer of the specimen and therefore does not reflect the true physical depth. To correct for such discrepancy, one needs to simply multiply the obtained layer equivalent air thickness by the refractive index of the layer material, to obtain the physical thickness. To demonstrate this, the inventors measured the thickness of 3 equal thickness layers, composed of different materials: silicon oxide, sapphire and silicon nitride, and plotted the relative measured thickness (FIG. 17 blue dots—three lower out of six dots). By multiplying the measured values by the refractive index of each material, the correct relative thickness (i.e. 1) was obtained (FIG. 17 red dots—three upper out of six dots).

### 5. Meta-Lens: Design and Expected Performances

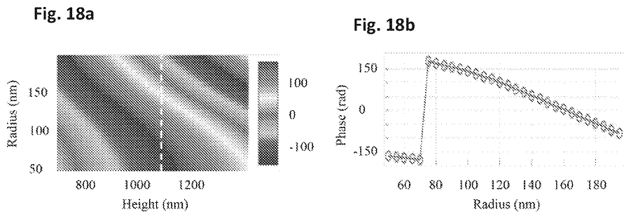

FIG. 18 shows: (a) Phase map at varying pillar heights (x axis) and radii (y axis). (b) A plot of the dashed line in (a), i.e. at a height of 1100 nm.

To design the meta-lens, the inventors used the commercial software PlanOpSim. First, a unit cell of 450 nm² composed of a silicon nitride pillar on a thick glass substrate was chosen; both the radius and the height of the pillar were swept to evaluate the phase delay for various parameter values. The result is shown in FIG. 18.

Ideally, the nanostructures composing the meta-lens should span a 2π phase variation to achieve the best focusing efficiency. However, due to fabrication constraints the inventors decided to avoid the smallest pillar diameters and settle for a range of ˜1.4 π which reduced the expected efficiency of the lens. Next, the full component was designed by choosing a phase profile (in this case the hyperbolic one as discussed above) and the software optimizes the nanopillars orientation to provide the desired phase distribution. The expected efficiency can be calculated by examining the total power at the near field region of the lens vs. the total power at the first order focal spot (an analysis tool which is built into the software). In this case the expected efficiency is ˜50% while the measured efficiency was ˜20%. The difference is attributed to fabrication discrepancies and scattering caused by surface roughness.

The drawings and the examples of the present disclosure present just some examples of the invented systems and methods. Many variations are possible within the scope of the disclosure, and they can provide ground to further claims than listed below.

1. A system for use in optical topographical and/or tomographic 3D imaging of a sample, comprising:
a lens unit, chromatically dispersive so that its focal length varies depending on a light wavelength, said lens unit being configured to pass therethrough polychromatic light arriving from and originated at a sample, while selectively collimating those spectral components of the polychromatic light which are in focus based on their wavelengths and origins, and
an etalon structure accommodated in an optical path of light being output from the lens unit to receive the collimated light, said etalon structure being configured to operate with multiple resonant wavelengths and to provide respective spectral transmittance peaks at said resonant wavelengths.

2. The system of claim 1, wherein said etalon structure has one of the following configurations:
the etalon structure is configured to provide simultaneous operation of said multiple resonant wavelengths; and
the etalon structure is tunable to operate with different resonance conditions each characterized by one of said multiple resonant wavelengths.

3. (canceled)

4. The system of claim 1, wherein the lens unit is characterized by at least one of the following:
the lens unit comprises a dispersive flat optical lens the lens unit comprises a diffractive zone plate and a refractive lens;
the lens unit at a nominal wavelength has a focal length in the range of 100 μm (microns) to 1 m;
the lens unit is configured to collect the polychromatic light from a field of view comprising angles of arrival up to 30°, or 10°, or 5° measured from an optical axis of the system;
the lens unit is configured to have a longitudinally chromatic aberration satisfying at least one from an inequality
$$\vert \frac{Δλ}{λ} \vert < 0.01 \vert \frac{Δ f}{f} \vert ,$$
an inequality
$$\vert \frac{Δλ}{λ} \vert < 0.1 \vert \frac{Δ f}{f} \vert ,$$
an inequality
$$\vert \frac{Δλ}{λ} \vert < 0.5 \vert \frac{Δ f}{f} \vert$$
and approximately an equality
$$\vert \frac{Δλ}{λ} \vert = \vert \frac{Δ f}{f} \vert ,$$
for two adjacent resonant wavelengths of the etalon structure at a normal incidence angle separated by a wavelength difference of Δλ, where the interval of Δλ between the two adjacent resonant wavelengths covers a nominal wavelength λ of the system, Δf is a difference between focal lengths of the dispersive lens unit at the two adjacent resonant wavelengths, and f is a focal length of the dispersive lens unit at the nominal wavelength of the system; and
the lens unit comprises an array of dispersive flat optical lenses.

5. The system of claim 4, wherein the lens unit comprises a dispersive flat optical lens, the dispersive flat optical lens having at least one of the following configurations: the dispersive flat optical lens is a diffractive lens; the dispersive flat optical lens is a meta-lens.

6-8. (canceled)

9. The system of claim 1, further comprising an optical detector configured to detect an output of the etalon structure consequential to said polychromatic light arriving from and originated at the sample and generate measured data indicative thereof.

10. The system of claim 9, wherein said optical detector has at least one of the following configurations:
the optical detector comprises a spectrophotometer;
the optical detector comprises an image sensor comprising a CCD image sensor or an active-pixel sensor;
the optical detector comprises a multispectral camera, optionally configured to operate with from 3 up to 30 spectral bands or other spectral modalities in each pixel, or a hyperspectral camera, optionally configured to operate with 30 to 200, or more, spectral bands or other spectral modalities in each pixel; and
said optical detector is configured to detect light with at least one wavelength being in a range from 300 nm to 1 mm.

11. (canceled)

12. The system of claim 9, wherein said optical detector comprises a multispectral camera, optionally configured to operate with from 3 up to 30 spectral bands or other spectral modalities in each pixel, or a hyperspectral camera, optionally configured to operate with 30 to 200, or more, spectral bands or other spectral modalities in each pixel, characterized by at least one of the following:
the spectral bands are distributed contiguously;
a free spectral range of the etalon structure is larger than a spectral resolution provided by the multispectral or hyperspectral camera; and
the lens unit is adapted to provide a longitudinally chromatic aberration so that the focal length changes in a spectrum detectable by the multispectral or hyperspectral camera by at least 1%, or 3%, or 10% of a nominal focal length.

13. (canceled)

14. The system of claim 13, wherein said other spectral modalities are modalities of time-domain Fourier transform imaging.

15-17. (canceled)

18. The system according to claim 9, comprising a control unit configured and operable to process the measured data and calculate a distance from the lens to a location at the sample, based on a spectral signal from said optical detector.

19. The system according to claim 9, wherein the optical detector comprises an image sensor comprising a CCD image sensor or an active-pixel sensor, the system comprising a control unit configured and operable to process the measured data and calculate a distance from the lens unit to a location at the sample based on a spectral signal from any one of the pixels of the image sensor of the optical detector.

20. The system of claim 18, wherein the control unit has at least one of the following configurations:
the control unit is configured to calculate the distance by taking into account also a spectral profile of light illuminating the sample;
the control unit is configured to calculate the distance based on a wavelength of an only one spectral band from spectral bands of the detector when the spectral signal represents a detection by said only one spectral band of a part of the polychromatic light originating at the location at the sample;
the control unit is configured to calculate the distance based on a wavelength of an only one spectral band from spectral bands of the detector when the spectral signal represents a detection by two spectral bands from the spectral bands of the detector of a part of the polychromatic light originating at the location at the sample, wherein the calculation is based on that spectral band which produced a relatively greater signal; and
the control unit is configured to calculate the distance by estimating a central wavelength of an envelope of a spectral distribution of a part of the polychromatic light originating at the location at the sample, when the spectral signal represents a detection by three or more of spectral bands of the detector of said part.

21-23. (canceled)

24. The system of claim 9, further comprising at least one of (a) an achromatic imaging lens system for directing that light output of the etalon structure which is to arrive to the optical detector, and (b) a reference arm optical detector and a reference arm achromatic imaging lens system for focusing a part of light reflected and/or emitted by the sample on the reference arm optical detector while bypassing the etalon structure.

25. The system of claim 1, wherein the etalon structure is characterized by at least one of the following:
the etalon structure a Fabry-Perot etalon;
a finesse of the etalon structure is in a range of from 10 to 150, or from 15 to 100, or from 25 to 75;
a free spectral range of the etalon structure is in a range of 10 nm-0.001 nm, or 10 nm-0.01 nm, or 10 nm-0.1 nm;
the etalon structure is tunable for adapting the resonant wavelengths thereof to a range of depths of the sample;
the etalon structure is configured with the multiple resonant wavelengths respectively varying for a range of incidence angles of collimated light on the etalon structure; and
the etalon structure is configured with the spectral transmittance peaks respectively varying for a range of incidence angles of collimated light on the etalon structure.

26-28. (canceled)

29. The system of claim 1, comprising a source of polychromatic illuminating light configured to illuminate a region at the sample to produce at least a part of the polychromatic light originating at the sample as a first or other order reflection and/or scattering and/or fluorescent and/or other response from an external surface and/or an internal surface and/or one or more depths of the sample.

30. The system of claim 29, characterized by at least one of the following:
the source of polychromatic illuminating light is adapted to provide broadband illumination comprising spectral components corresponding to a plurality of the resonant wavelengths of the etalon structure;
the source of polychromatic illuminating light is adapted to provide illumination with a spectral intensity distribution peaking at a plurality of the resonant wavelengths of the etalon structure;
the system comprises a machine-readable memory storing a record on a predetermined or measured spectral profile of the polychromatic illuminating light for determining a spectral profile of light illuminating the sample;
the system comprises at least one polarizing unit, accommodated in an optical path of the illuminating light to the sample and/or in an optical path of the collimated light to the etalon structure, the at least one polarizing unit being configured to provide to light passing therethrough a TE-mode, or a TM mode, or a circular polarization;
the system comprises an optical splitter accommodated in an optical path of light output from the etalon structure and configured to split from it the consequential output of the etalon structure; and
the system comprises a spectrometer accommodated to detect a spectral distribution of said consequential output of the etalon structure.

31-41. (canceled)

42. The system of claim 1, comprising a support stage for supporting a sample under measurements, the system being configured and operable to affect a relative displacement in at least one lateral dimension between said stage and an optical unit formed by the dispersive flat lens unit and the etalon structure.

43. An optical unit for use in a microscope, the optical unit comprising the system of claim 1.

44. A method for use in optical topographical and/or tomographic 3D imaging of a sample, comprising:
passing through a lens unit, chromatically dispersive so that its focal length varies depending on a light wavelength, polychromatic light arriving from and originated at a sample, while selectively collimating those spectral components of the polychromatic light which are in focus based on their wavelengths and origins, and receiving the collimated light at an etalon structure, accommodated in an optical path of light being output from the lens unit and configured to operate with multiple resonant wavelengths to provide respective spectral transmittance peaks at said resonant wavelengths to the collimated light.

45. The method of claim 44, comprising passing a part of the collimated light though the etalon structure.

46. The method of claim 44, further comprising detecting an output of the etalon structure consequential to said polychromatic light arriving from and originated at the sample, and generating measured data indicative of said output, with an optical detector.

47. The method of claim 46, further comprising processing with a control unit the measured data to calculate a distance from the lens to a location at the sample, based on a spectral signal from said optical detector.

48. A non-transitory machine-readable medium storing instructions executable by a processor, the non-transitory machine-readable medium comprising:
instructions to calculate with the measured data generated by the method of claim 46 a distance from the lens to a location at the sample, based on a spectral signal from said optical detector.

49. A system for use in optical topographical and/or tomographic 3D imaging of a sample, comprising:
a lens unit, chromatically dispersive so that its focal length varies depending on a light wavelength, said lens unit being configured to pass therethrough polychromatic light arriving from and originated at a sample, while selectively collimating those spectral components of the polychromatic light which are in focus based on their wavelengths and origins,
an etalon structure accommodated in an optical path of light being output from the lens unit to receive the collimated light, said etalon structure being configured to operate with multiple resonant wavelengths and to provide respective spectral transmittance peaks at said resonant wavelengths; and
an optical detector configured to detect an output of the etalon structure consequential to said polychromatic light arriving from and originated at the sample and generate measured data indicative thereof, thereby enabling to determine, from said measured, a distance from the lens to a location at the sample, based on a spectral signal from said optical detector.
